(19) United States
(12) Patent Application Publication
(10) Pub. No.: US 2010/0167040 A1
(43) Pub. Date: Jul. 1, 2010

(54) IDENTIFICATION OF A NOVEL TYPE OF SUCROSE SYNTHASE AND USE THEREOF IN FIBER MODIFICATION

(75) Inventors: Yong Ling Ruan, New South Wales (AU); Robert Furbank, New South Wales (AU); Elizabeth Brill, Australian Capital Territory (AU); Michel Van Thournout, Sint-Michiels (BE); Antonio Arioli, Lubbock, TX (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignees: BAYER BIOSCIENCE N.V., BELGIUM (BE); COMMONWEALTH SCIENTIFIC AND INDUSTRIAL RESEARCH ORGANIZATION, CAMPBELL (AU)

(21) Appl. No.: 12/374,965

(22) PCT Filed: Jul. 20, 2007
(86) PCT No.: PCT/EP2007/006551

§ 371 Date: Nov. 19, 2009

Related U.S. Application Data
(60) Provisional application No. 60/834,066, filed on Jul. 28, 2006.

(30) Foreign Application Priority Data
Jul. 25, 2006 (EP) 06015433.3

(51) Int. Cl. D04H 1/02; A01H 5/00; C12N 15/82; C12N 9/10; C07K 16/00; C07H 21/04; C12N 15/74; C12N 5/10

(52) U.S. Cl. 428/292.1; 800/298; 800/314; 800/284; 435/193; 530/387.1; 536/23.2; 435/320.1; 435/419; 442/327

(57) ABSTRACT

Methods and means are described to modify fiber characteristics in a fiber producing plant, such as cotton, based on the use of a novel type of sucrose synthase protein or related nucleic acids.

### FIELD OF THE INVENTION

The invention relates to the field of agriculture, more specifically towards the use of molecular biology techniques to alter fiber producing plants, particularly cotton plants and/or accelerate breeding of such fiber containing plants. Methods and means are provided to modify fiber quality e.g. by increasing or decreasing cellulose contents, fiber strength, fiber uniformity or micronaire. Methods are also provided to identify molecular markers associated with such characteristics in a population of cotton varieties and related progenitor plants.

### BACKGROUND ART

Much of the high quality fiber for the textile industry is provided for by cotton. About 90% of cotton grown worldwide is Gossypium hirsutum L., whereas Gossypium barbadense accounts for about 8%. Consequently, the modification of cotton fibers characteristics to better suit the requirements of the industry is a major effort in breeding by either classical methods or by genetically altering the genome of cotton plants. Goals to be achieved include increased lint fiber length, strength, dyability decreased fuzz fiber production, fiber maturity ratio, immature fiber content, fiber uniformity and micronaire.

U.S. Pat. No. 6,472,588 and WO0117333 provides methods for increasing the quality of cotton fiber produced from a cotton plant by transformation with a DNA encoding sucrose phosphate synthase. The fiber qualities include strength, length, fiber maturity ratio, immature fiber content, fiber uniformity and micronaire.

WO9508914 discloses a fiber producing plant comprising in its genome a heterologous genetic construct. The genetic construct comprises a fiber-specific promoter and a coding sequence encoding a plant peroxidase, such as a cotton peroxidase.

WO9626639 provides methods whereby encoding sequence preferentially directing gene expression in ovary tissue, particularly very early in fruit development, are utilized to express plant growth modifying hormones in cotton ovule tissue. The methods permit the modification of the characteristics of boll set in cotton plants and provide a mechanism for altering fiber quality characteristics such as fiber dimension and strength.

U.S. Pat. No. 5,981,834, U.S. Pat. No. 5,597,718, U.S. Pat. No. 5,620,882, U.S. Pat. No. 5,521,708 and U.S. Pat. No. 5,495,070 all disclose a method for genetically engineering a fiber-producing plant and the identification of cDNA clones useful for identifying fiber genes in cotton. The cDNA clones are useful in developing corresponding genomic clones from fiber producing plants to enable genetic engineering of cotton and other plants using these genes. Coding sequence from these isolated genes are used in sense or antisense orientation to alter the fiber characteristics of transgenic fiber producing plants.

Published US patent applications US2002049999 and US2003074697 both disclose cotton plants of the genus Gossypium with improved cotton fiber characteristics. The cotton plant has an expression cassette containing a gene coding for an enzyme selected from the group consisting of endoxyloglucan transferase, catalase and peroxidase so that the gene is expressed in cotton fiber cells to improve the cotton fiber characteristics.

U.S. Pat. No. 5,880,110 produces cotton fibers with improved fiber characteristics by treatment with brassinosteroids.

WO 01/40250 provides methods for improving cotton fiber quality by modulating transcription factor gene expression.

WO 96/40924 provides novel DNA constructs which may be used as molecular probes or inserted into a plant host to provide for modification of transcription of a DNA sequence of interest during various stages of cotton fiber development. The DNA constructs comprise a cotton fiber transcriptional initiation regulatory region associated with a gene, which is expressed in cotton fiber. Also is novel cotton having a cotton fiber, which has a natural color, introduced by the expression in cotton fiber cell, using such a construct, of pigment genes.

EP0834566 provides a gene which controls the fiber formation mechanism in cotton plant and which can be used for industrially useful improvement.

Ruan et al. 2003 (The Plant Cell, vol 15, 952-964) describe that suppression of sucrose synthase gene expression represses cotton fiber cell initiation, elongation and seed development. The results described herein provide direct evidence that the expression of Sucrose synthase in the ovule epidermis is crucial for the initiation and elongation of single-celled fibers. The described results also allow to conclude that the suppression of Sus in the seed coat only reduces fiber growth without affecting embryo development and seed size. The article concludes that the novel insights in the role of Sus in controlling plant cell and seed development offer opportunities to modify fiber and seed development through genetic engineering of Sus expression.

WO0245485 describes methods and means to modulate fiber quality in fiber-producing plants, such as cotton, by modulating sucrose synthase activity and/or expression in such plants.

Baud et al. 2004 (Journal of Experimental Botany, Vol 55, No. 396, pp 397-409) describe the structure and expression profile of the sucrose synthase multigene family having six members in Arabidopsis.

None of the prior art document describe the recognition of the currently recognized type of sucrose synthase, nor its involvement in secondary plant cell wall development, such as the type C in plants. This novel type of Sus protein opens the possibility for new and improved methods and means to alter fiber characteristics of fiber-producing plants such as cotton, which may be further combined with any of other methods to alter fiber characteristics. Such methods and means are described in the embodiments and claims described hereinafter.

### SUMMARY OF THE INVENTION

In one embodiment of the invention, a novel type of sucrose synthase protein is provided having an amino acid sequence comprising an amino acid sequence selected from an amino acid sequence having at least 50% sequence homology to the amino acid sequence of any one of SEQ ID Nos.: 2, 3, 5, 7, 9, 11 or 13; an amino acid sequence comprising the amino acid sequence of any one of SEQ ID Nos.: 2, 3, 5, 7, 9, 11 or 13; an amino acid sequence located in the amino-terminal part of said protein, said amino acid sequence comprising at least about 60% sequence identity to the amino acid sequence of SEQ ID No. 16; or an amino acid sequence located at the carboxy-terminal part of said protein, said amino acid sequence comprising at least about 60% sequence identity to the amino acid sequence of SEQ ID No. 15. The sucrose synthase protein may comprise a hydrophobic N-terminal sequence. It may also further comprise any one of the following amino acid sequences: the amino acid sequence of SEQ ID No.: 3 from amino acid 383 to amino acid 394; the amino acid sequence of SEQ ID No.: 3 from amino acid 270 to amino acid 329; the amino acid sequence of SEQ ID No.: 3 from amino acid 549 to amino acid 737; the amino acid sequence of SEQ ID No.: 3 from amino acid 1 to amino acid 545; or the amino acid sequence of SEQ ID No.: 3 from amino acid 18 to amino acid 794.

In another embodiment of the invention, an antibody recognizing the isolated novel sucrose synthase protein is provided.

In yet another embodiment, the invention provides an isolated DNA molecule or nucleic acid encoding the novel sucrose synthase protein, such as nucleic acid comprising the nucleotide sequence of any one of SEQ ID Nos.: 1, 4, 6, 8, 10, 12 or 14.

The invention further provided an expression cassette comprising the following operably linked DNA molecules: a plant expressible promoter such as a plant expressible promoter controlling transcription preferentially in fiber cells of a fiber producing plant; a DNA encoding the novel sucrose synthase proteins provided; and optionally a transcription termination and polyadenylation region.

The invention also provides an expression cassette comprising the following operably linked DNA molecules: a plant expressible promoter a DNA which when transcribed results in an RNA molecule said RNA molecule comprising either a nucleotide sequence of at least 19 consecutive nucleotides having at least about 94% sequence identity to the nucleotide sequence of an endogenous sucrose synthase isoform C encoding gene or to the complement of said nucleotides sequence of an endogenous sucrose synthase isoform C encoding gene or a nucleotide sequence of at least 19 consecutive nucleotides having at least about 94% sequence identity to a nucleotide sequence of novel type C isoform sucrose synthases; and optionally a transcription termination and polyadenylation region.

Another embodiment of the invention is an expression cassette comprising the following operably linked DNA molecules: a plant expressible promoter, preferably a plant expressible promoter which controls transcription preferentially in the fiber cells; a DNA, which when transcribed yields a double-stranded RNA molecule capable of reducing the expression of a SusC gene endogenous to the fiber producing plant, and the RNA molecule comprising a first and second RNA region wherein the first RNA region comprises a nucleotide sequence of at least 19 consecutive nucleotides having at least about 94% sequence identity to the nucleotide sequence of an endogenous SusC gene or to the nucleotide sequence of the provided nucleotide sequences encoding SusC proteins; the second RNA region comprising a nucleotide sequence complementary to the at least 19 consecutive nucleotides of the first RNA region; the first and second RNA region are capable of base-pairing to form a double stranded RNA molecule between at least the 19 consecutive nucleotides of the first and second region; and optionally a 3′ end region comprising transcription termination and polyadenylation signals functioning in cells of the plant.

The invention further provides a plant cell comprising a heterologous plant expressible promoter operably linked to a DNA molecule selected from the following DNA molecules: a DNA encoding a novel sucrose synthase of the C isoform or encoding a inhibitory RNA for a susC encoding gene. The cell may be a cell from a fiber producing plant such as cotton. Also encompassed by the invention are plants, seeds or plant parts or tissues which comprise or consist essentially of such plant cells as well as fibers produced by such plants.

In yet another embodiment of the invention, a method for modifying the fiber characteristics of a fiber-producing plant is provided, said method comprising modifying, such as increasing or decreasing the functional level of a sucrose synthase isoform C or a sucrose synthase with similar characteristics in cells or cell walls or apoplastic fluid of said plant. This can be conveniently achieved by providing the plants with the expression cassettes described herein.

The invention also encompasses use of a novel SusC type protein or encoding nucleic acid to modify the characteristics of a fiber in a fiber producing plant.

### BRIEF DESCRIPTION OF THE FIGURES AND DRAWINGS

FIG. 1. Hydrophobicity plots of SusC proteins and similar sucrose proteins. A. Plot for SusC from a Gossypium hirsutum variety; B: Plot for SusC from a Gossypium barbadense variety; C: Plot for SusC from Gossypium arboretum; D: Plot for SusC from Gossypium raymondii; E: Plot for SusC from Gossypium longicalyx subtype 1; F: Plot for SusC from Gossypium longicalyx subtype 2; G: Plot for sucrose synthase from Vigna radiata; H: Plot for sucrose synthase from Eucalyptus grandis; I: Plot for sucrose synthase isoform 6 from Arabidopsis thaliana; K: Plot for sucrose synthase from Poplins tremuloides. All hydrophobicity plots reveal a hydrophobic N-terminus for the different sucrose synthases.

FIG. 2. Expression analysis of the SusC gene in cotton fiber tissue by RT-PCR. Dpa: days post anthesis; gDNA: control genomic DNA. Lanes 1 and 8: 1 kb marker.

FIG. 3: Intron and exons in sucrose synthase encoding genes from cotton. The intron exon distribution is schematically represented for sucrose synthase type C and compared with type B and A sucrose synthase from cotton. Black boxes represent the exons.

FIG. 4: 2D structure analysis of the N-terminal part of sucrose synthase genes from cotton. 2D structure analysis (HCA+JPRED) of the N-terminal part of the susyC gene compared to the other Gossypium hirsutum sucrose synthases. The arrow shows the influence of the susyC specific region on the surrounding 2D-structure. (P)=putative phosphorylation sites.

FIG. 5: 2D structure analysis of the C-terminal part of sucrose synthase genes from cotton. The beta-sheet structure seems to be absent in the susyC 2D structure.

FIG. 6: Phenotypic analysis of transgenic Arabidopsis plants. Thickened, bifurcated, fasciated stems are shown in 2 independent transgenic lines harboring CaMV 35S-SusC chimeric genes (panels A and B). Panels C and D display a typical trichome phenotype in the T2 generation of CaMV 35S-SusC and S2A-SusC chimeric genes. Panel C is a 3-dimensional photo-montage.

FIG. 7: Western blot of T2 plantlets with CaMV 35S-SusC transgene using SUS C specific antibody. Lane A: plants with mild phenotype; Lane B: plant with severe phenotype, Lane C: plant with mild phenotype; Lane D: plant with severe phenotype; WT: wild type plant.

### DETAILED DESCRIPTION

The current invention is based on the identification of a new sucrose synthase isoform (named SusC or SuSyC) from fibers whose expression profile at the RNA level is closely correlated with the onset of secondary cell wall development phase in fiber-producing plants such as cotton. The protein is abundant during the secondary cell wall formation and is located predominantly in the apoplast. Its location, abundance and expression profile indicate that this novel type of sucrose synthase isoform is the major isoform present in the cell wall at the secondary cell wall synthesis stage. Without intending to limit the invention to a particular mode of action, it is thought that this sucrose synthase isoform is involved in scavenging sugars from the apoplastic fluid surrounding the fiber and incorporating them as UDPGlucose into cellulose and/or callose.

Accordingly, in a first embodiment, the invention provides a method for modifying cell wall characteristics in a plant cell characterized in that the functional level of this novel isoform of sucrose synthase (SusC) or sucrose synthases with similar characteristics, is modified.

The functional level of the sucrose synthase type C isoform, or sucrose synthases with similar characteristics may be modified by increased expression of such an isoform in the plant cell. This may be conveniently achieved by introduction of an expression construct comprising the following operably linked nucleic acids, e.g. DNA molecules:

a) a plant-expressible promoter

b) a nucleic acid encoding a sucrose synthase isoform C or a sucrose synthase with similar characteristics; and optionally

c) a transcription termination and polyadenylation region.

As used herein, “sucrose synthase” refers to an enzyme that is capable of catalyzing the synthesis of sucrose from NDP-glucose (such as uridine diphosphate glucose) and D-fructose. The enzyme may also catalyze the hydrolysis of sucrose in glucose and fructose.

The enzyme is classified as EC 2.4.1.13. Synonyms for sucrose synthase are glucosyltransferase, uridine diphosphoglucose-fructose, sucrose synthetase, sucrose-UDP glucosyltransferase, sucrose-uridine diphosphate glucosyltransferase, Sus, SuSy, UDP-glucose-fructose glucosyltransferase, UDP-glucose:D-fructose 2-alpha-D-glucosyltransferase, and uridine diphosphoglucose-fructose glucosyltransferase.

Several genes from plants encoding sucrose synthase enzymes have been described. WO02/45485 (pages 16 to 20) provides an extensive list of Genbank accession numbers for nucleotide sequence encoding plant sucrose synthase genes, parts thereof or nucleotide sequences having sequence similarity to sucrose synthase genes.

Enzymatic assays for sucrose synthase activity have been described (see e.g. Counce and Gravois, 2006, Crop Science Vol 46: pp 1501-1507, particularly pages 1502 and 1503; Anthon and Emerich, 1990, Vol 92: pp 346-351, particularly page 347; both documents herein incorporated by reference).

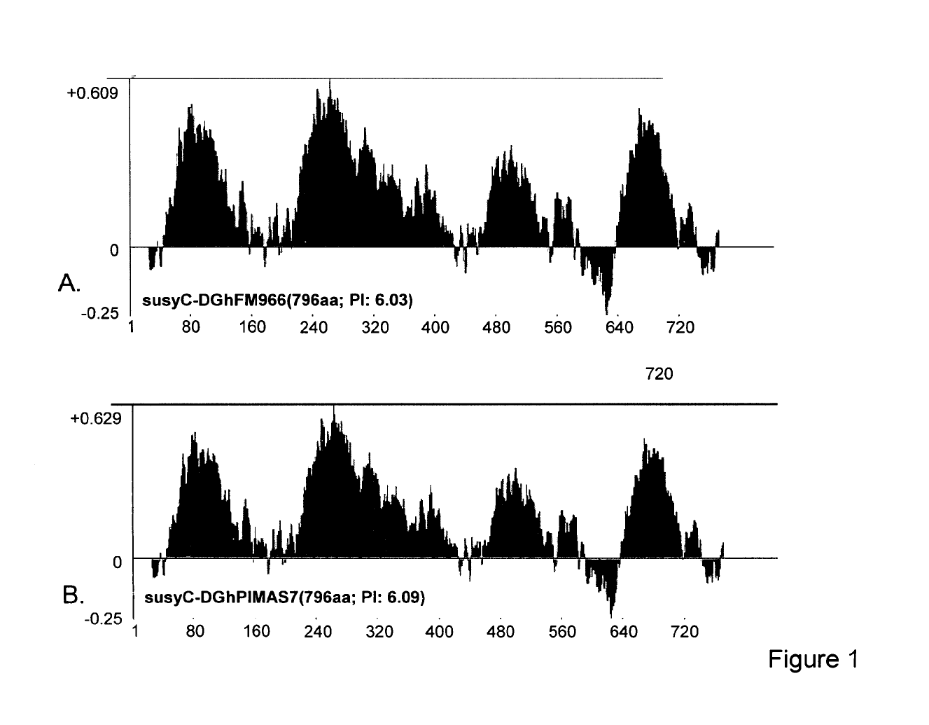

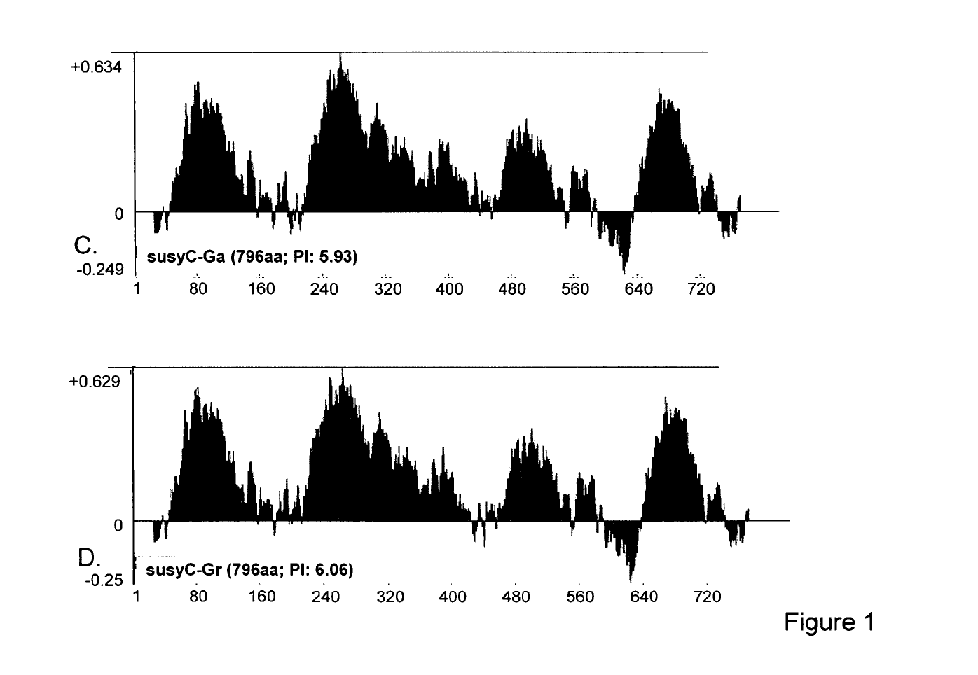

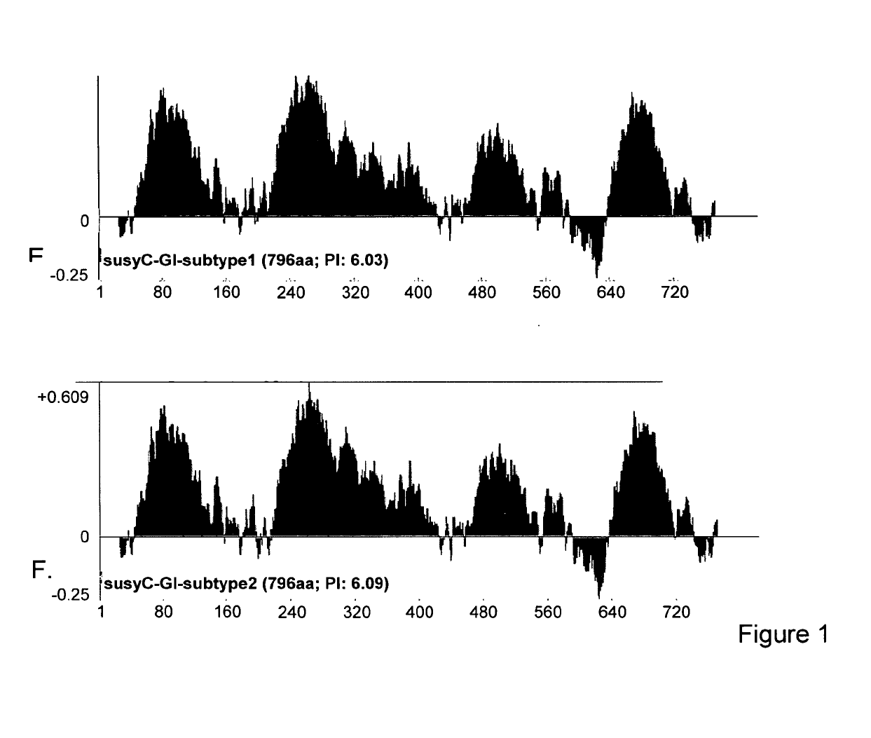

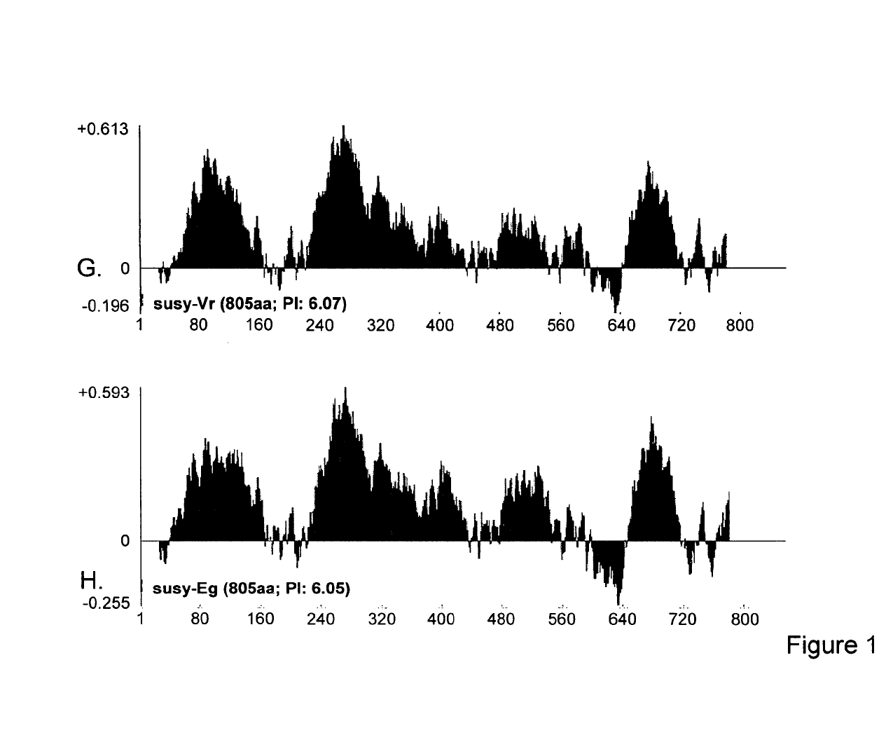

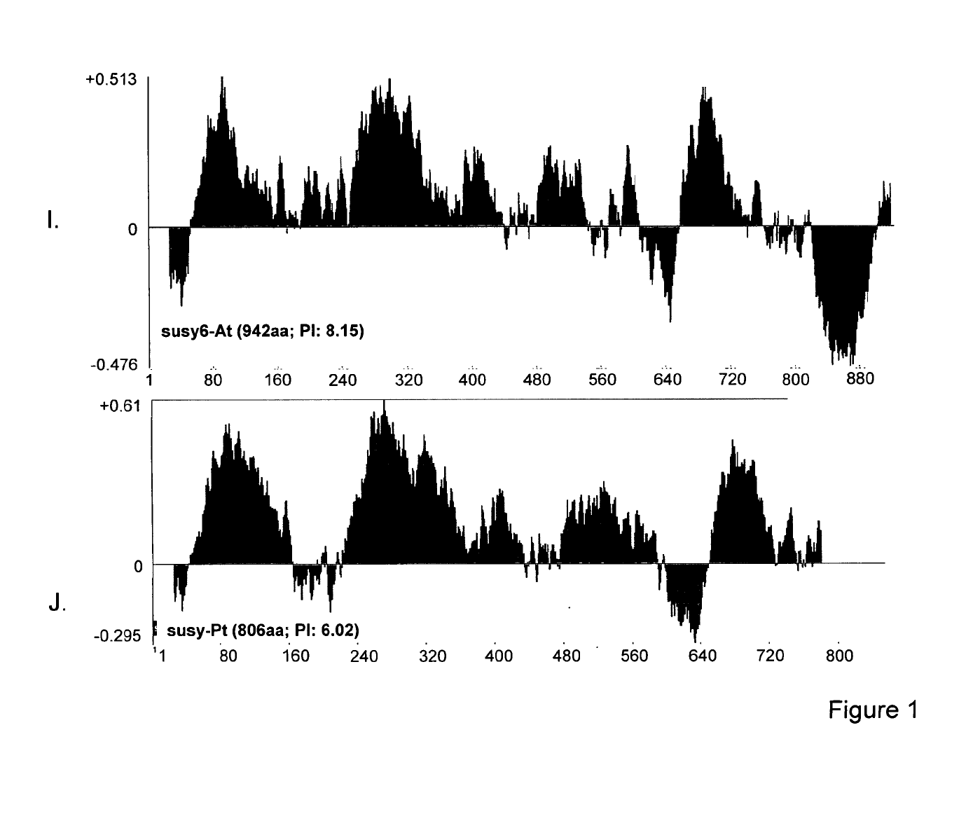

The “sucrose syntase isoform C” or “SusC” or “SuSyC” is characterized by the presence of a hydrophobic N-terminal amino acid sequence (See FIG. 1). The first 44 amino acids form a hydrophobic region, particularly residues 26 to 44 of e.g. SEQ ID No.:3. Other plant sucrose synthase enzymes characterized by such a hydrophobic N-terminal amino acid sequence are sucrose synthase 6 from Arabidopsis thaliana (Accession number At1g73370); the sucrose synthase from mungbean (Accession number D10266), the sucrose synthase from Eucalyptus spp. (Accession number DD014303) and the sucrose synthase from poplars (Accession number AY341026) (all sequences herein incorporated by reference).

Alignment of the different SusC amino acid sequences and comparison with other sucrose synthases from plants revealed the presence of stretches of 20-25 consecutive amino acids located at the N-terminus (SEQ ID No:16) or C-terminus (SEQ ID No. 15) respectively, which are conserved within in the SusC amino acid sequences and absent from the other plant sucrose synthases. The N-terminal sequence (HKSQKLLSVLDKEAGNQALDGMVV; SEQ ID No.:16) is usually located between amino acid position 36 and amino acid position 59, while the C-terminal sequence (AYQEQRGRKRYIEMLHAWMYNNRVKT; SEQ ID No.: 15) is usually located between amino acid positions 765 and 790.

Sucrose synthase proteins have also been described to possess a putative actin binding region (Winter H. et al., 1998, FEBS letters 430, 205-208; Winter H. and Huber S.C., 2000 Critical Reviews in Plant Sciences 19(1), 31-67) which in SusC isoforms has the following consensus amino acid sequence: KDVAAE[V/I]TKEFQ (SEQ ID No 3 from amino acid position 383 to amino acid position 394). The lysine residue (K) usually found at position 383 and the phenylalanine residue (F), usually found at position 393, are also indicative of a SusC type protein.

Sucrose synthase proteins have also been described to possess a putative Uridine binding region which in SusC isoforms has the following amino acid sequence VVIMTPHGYFAQDNVLGYPDTGGQVVYILDQVRALEEELLHRFKLQGLDITPRIL VITRL (SEQ ID No 3 from amino acid position 270 to amino acid position 329).

Using computer based searches the following domains or signatures could be identified in the amino acid sequence of the SusC isoforms. These domains or signatures are shared with other sucrose synthases from plants.

Glycosyl Transferase, Group 1 Domain: (IPRO01296; PF00534)

Proteins containing this domain transfer UDP, ADP, GDP or CMP linked sugars to a variety of substrates, including glycogen, fructose-6-phosphate and lipopolysaccharides. The bacterial enzymes are involved in various biosynthetic processes that include exopolysaccharide biosynthesis, lipopolysaccharide core biosynthesis and the biosynthesis of the slime polysaccaride colanic acid. This domain corresponds to the amino acid sequence of SEQ ID No 3 e.g from amino acid position 549 to 737.

Sucrose synthase family signature (IPR000368; PF00862): This signature characterizes a family including the bulk of the sucrose synthase proteins. However the carboxyl terminal region of the sucrose synthases belongs to the glycosyl transferase family. This enzyme is found mainly in plants but also appears in bacteria. This domain corresponds to the amino acid sequence of SEQ ID No 3 e.g from amino acid position 1 to 545

Sucrose Synthase, Plants and Cyanobacteria Family Signature (IPRO12820)

This signature represents sucrose synthases an enzyme that despite its name, generally uses rather produces sucrose. Sucrose plus UDP (or ADP) becomes D-fructose plus UDP-glucose (or ADP-glucose), which is then available for cell wall (or starch) biosynthesis. The enzyme is homologous to sucrose phosphate synthase, which catalyses the penultimate step in sucrose synthesis. Sucrose synthase is found, so far, exclusively in plants and cyanobacteria. This domain corresponds to the amino acid sequence of SEQ ID No 3 e.g from amino acid position 18 to 794.

These domains can easily be recognized by computer based searches using e.g. PROSITE profiles (PROSITE is available at www.expasy.ch/prosite). Alternatively, the BLOCKS database and algorithm (blocks.fhcrc.org) may be used to identify these domains. Other databases and algorithms are also available (pFAM: http://www.sanger.ac.uk/Software/Pfam/ INTERPRO: http://www.ebi.ac.uk/interpro/; the above cited PF numbers refer to pFAM database and algorithm and IPR numbers to the INTERPRO database and algorithm).

The current invention provides a number of variant amino acid sequences and nucleotide sequences (cDNA as well as genomic DNA) for SusC isoforms isolated from Gossypium hirsutum cultivars (SEQ ID Nos: 3-4), Gossypium barbadense cv (SEQ ID Nos: 5 and 6), Gossypium arboretum (SEQ ID Nos: 7 and 8), Gossypium raimondii (SEQ ID Nos: 9 and 10) and Gossypium longicalyx (SEQ ID Nos.: 11 to 14).

Other variant nucleotide sequences may be obtained from other Gossypium hirsutum or Gossypium barbadense cultivars or from cotton progenitor plants such as Gossypium arboretum, Gossypium herbaceum and Gossypium raimondii and Gossypium longicalyx, or other Gossypium species, especially Gossypium species comprising a D-type genome, such as Gossypium aridum, Gossypium davidsonii, Gossypium gossypioides, Gossypium klotzschianum, Gossypium turberi, Gossypium trilobum, Gossypium turneri, Gossypium mustelinum, Gossypium tomentosum, Gossypium darwinii. Variant amino acid sequences or nucleotide sequences include modifications of a sequence by addition, deletion or substitution of amino acids or nucleotides, respectively. PCR amplification of SusC encoding nucleic acids is possible from other Gossypium species comprising a D-type genome. Such PCR amplified nucleic acids appear to comprise an EcoRI restriction site characteristic for SusC type encoding nucleic acids.

Variants of the sucrose synthases isoform C may be found by stringent hybridization using the nucleotide sequence of any one of SEQ ID No 1, 4, 6, 8, 10, 12, or 14 or a part thereof comprising at least about 25 or 50 consecutive nucleotides of SEQ ID No SEQ ID No 1, 4, 6, 8, 10, 12 or 14 or the complementary nucleotide sequences thereof, as a probe. A particular useful probe can be the nucleotide sequence encoding the N-terminal or C-terminal sequence of SEQ ID 15 and 16, such as the nucleotide sequences of any one of SEQ ID No 1, 4, 6, 8, 10, 12 or 14 encoding the amino acid sequence of SEQ ID Nos 15 or 16.

“Stringent hybridization conditions” as used herein means that hybridization will generally occur if there is at least 95% and preferably at least 97% sequence identity between the probe and the target sequence. Examples of stringent hybridization conditions are overnight incubation in a solution comprising 50% formamide, 5×SSC (150 mM NaCl, 15 mM trisodium citrate), 50 mM sodium phosphate (pH 7.6), 5× Denhardt's solution, 10% dextran sulfate, and 20 μg/ml denatured, sheared carrier DNA such as salmon sperm DNA, followed by washing the hybridization support in 0.1×SSC at approximately 65° C., preferably for about 10 minutes. Other hybridization and wash conditions are well known and are exemplified in Sambrook et al, Molecular Cloning: A Laboratory Manual, Second Edition, Cold Spring Harbor, N.Y. (1989), particularly chapter 11.

Such variant sequences may also be obtained by DNA amplification using oligonucleotides specific for sucrose synthase (SusC) genes as primers, such as but not limited to oligonucleotides comprising or consisting of about 20 to about 50 consecutive nucleotides of the nucleotide sequence of SEQ ID No 1, 4, 6, 8, 10, 12 or 14 or their complement. SEQ ID Nos 17 and 18 set forth the nucleotide sequences of primers which are particularly suited as SusC specific primers.

In addition to exploiting the natural variation for SusC isoforms, variant sequences may also be obtained by induced generation of variation in vitro or in vivo. Several methods for in vitro induced generation of variant nucleotide sequences are available in the art including but not limited to DNA shuffling or directed evolution techniques as described in U.S. Pat. No. 5,605,793, U.S. Pat. No. 5,811,238 and U.S. Pat. No. 5,830,721. Methods for in vivo induced generation of variant nucleotide sequence are also well known in the art and may include exposure of cotton plants or cotton progenitor plants to mutagens such as ionizing radiation, EMS, MMS or the like, followed by isolation of the SusC encoding nucleic acids, e.g. as elsewhere described herein.

Having identified the C-terminal and N-terminal consensus sequences for a SusC type sucrose synthase, the inventors have identified the following nucleotide sequences in databases encoding amino acid sequences comprising such consensus sequences. Using the N-terminal consensus region (SEQ ID No.:16) the following sequences (identified by their Accession number) were isolated: EMBL:CO499214 (Gossypium hirsutum cDNA); EMBL:CO499090 (Gossypium hirsutum cDNA); EMBL:CO498472 (Gossypium hirsutum cDNA); EMBL:CO498387 (Gossypium hirsutum cDNA); EMBL:CO497432 (Gossypium hirsutum cDNA); EMBL:CO497275 (Gossypium hirsutum cDNA); EMBL:CO496887 (Gossypium hirsutum cDNA); EMBL:CO496593 (Gossypium hirsutum cDNA); EMBL:CO495954 (Gossypium hirsutum cDNA); EMBL:CO495804 (Gossypium hirsutum cDNA); EMBL:CO495643 (Gossypium hirsutum cDNA); EMBL:CO495623 (Gossypium hirsutum cDNA); EMBL:CO495601 (Gossypium hirsutum cDNA); EMBL:CO495511 (Gossypium hirsutum cDNA); EMBL:CO494831 (Gossypium hirsutum cDNA); EMBL:CO494779 (Gossypium hirsutum cDNA); EMBL:CO494693 (Gossypium hirsutum cDNA); EMBL:CO494536 (Gossypium hirsutum cDNA); EMBL:CO494182 (Gossypium hirsutum cDNA); EMBL:CO0493935 (Gossypium hirsutum cDNA); EMBL:CO493780 (Gossypium hirsutum cDNA); EMBL:CO493725 (Gossypium hirsutum cDNA); EMBL:CO493724 (Gossypium hirsutum cDNA); EMBL:CO493589 (Gossypium hirsutum cDNA); EMBL:CO493043 (Gossypium hirsutum cDNA); EMBL:CO493007 (Gossypium hirsutum cDNA); EMBL:CO492969 (Gossypium hirsutum cDNA); EMBL:CO492566 (Gossypium hirsutum cDNA); EMBL:CO490959 (Gossypium hirsutum cDNA); EMBL:BF272227 (Gossypium arboreum cDNA clone GA_Eb0014E15f); EMBL:CO493732 (Gossypium hirsutum cDNA); EMBL:CO493111 (Gossypium hirsutum cDNA); EMBL:CO491540 (Gossypium hirsutum cDNA); EMBL:CO493583 (Gossypium hirsutum cDNA); EMBL:CO493765 (Gossypium hirsutum cDNA); and EMBL:CO498054 (Gossypium hirsutum cDNA). All cited sequences are herein incorporated by reference. Using the C-terminal consensus region (SEQ ID No.:15) the following sequences (identified by their Accession number) were isolated: EMBL:DT463218 (Gossypium hirsutum cDNA clone GH_CHX13D22-3); EMBL:CO499051 (Gossypium hirsutum cDNA); EMBL:CO498850 (Gossypium hirsutum cDNA); EMBL:CO498685 (Gossypium hirsutum cDNA); EMBL:CO498361 (Gossypium hirsutum cDNA); EMBL:CO498044 (Gossypium hirsutum cDNA); EMBL:CO497702 (Gossypium hirsutum cDNA); EMBL:CO497506 (Gossypium hirsutum cDNA); EMBL:CO497291 (Gossypium hirsutum cDNA); EMBL:CO497151 Gossypium hirsutum cDNA); EMBL:CO496924 (Gossypium hirsutum cDNA); EMBL:CO496907 (Gossypium hirsutum cDNA); EMBL:CO496748 (Gossypium hirsutum cDNA); EMBL:CO496747 (Gossypium hirsutum cDNA); EMBL:CO496373 (Gossypium hirsutum cDNA); EMBL:CO496003 (Gossypium hirsutum cDNA); EMBL:CO495974 (Gossypium hirsutum cDNA); EMBL:CO495570 (Gossypium hirsutum cDNA); EMBL:CO494691 (Gossypium hirsutum cDNA); EMBL:CO493817 (Gossypium hirsutum cDNA); EMBL:CO493779 (Gossypium hirsutum cDNA); EMBL:CO493750 (Gossypium hirsutum cDNA); EMBL:CO492925 (Gossypium hirsutum cDNA); EMBL:CO492917 (Gossypium hirsutum cDNA); EMBL:CO492529 (Gossypium hirsutum cDNA); EMBL:CO492496 (Gossypium hirsutum cDNA); EMBL:CO493902 (Gossypium hirsutum cDNA); EMBL:BQ412019 Gossypium arboreum cDNA clone GA_Ed0053A09r); EMBL:CO497086 (Gossypium hirsutum cDNA) EMBL:CO494597 (Gossypium hirsutum cDNA); EMBL:CO494374 (Gossypium hirsutum cDNA); EMBL:CO499615 (Gossypium hirsutum cDNA); EMBL:CO490812 (Gossypium hirsutum cDNA); EMBL:CO493178 (Gossypium hirsutum cDNA). All cited sequences are herein incorporated by reference. Although none of these sequences encodes a complete SusC protein, the different parts may be used as probes to identify further SusC proteins. The different parts of the cDNA may also be used to reconstitute a C-type sucrose synthase encoding nucleic acid.

Variant forms of sucrose synthase isoform C suitable for the invention may thus have a amino acid sequence which comprises an amino acid sequence having at least about 60% or about 70% or about 80% or about 90% or about 95% sequence identity to the amino acid sequence of N-terminal consensus sequence (SEQ ID No 16) or the C-terminal consensus sequence (SEQ ID No 15) or both. The variants may have and amino acid sequence having at least about 60% or about 70% or about 80% or about 90% or about 95% sequence identity to the amino acid sequence of any one of the SusC isoforms of SEQ ID Nos.: 2, 3, 5, 7, 9, 11 or 13. Nucleotide sequences encoding such variants may have a nucleotide sequence having at least about 60% or about 70% or about 80% or about 90% or about 95% sequence identity to the nucleotide sequence of any one of the nucleotide sequences of SEQ ID Nos.: 1, 4, 6, 8, 10, 12 or 14.

For the purpose of this invention, the “sequence identity” of two related nucleotide or amino acid sequences, expressed as a percentage, refers to the number of positions in the two optimally aligned sequences which have identical residues (×100) divided by the number of positions compared. A gap, i.e. a position in an alignment where a residue is present in one sequence but not in the other, is regarded as a position with non-identical residues. The alignment of the two sequences is performed by the Needleman and Wunsch algorithm (Needleman and Wunsch 1970). The computer-assisted sequence alignment above, can be conveniently performed using standard software program such as GAP which is part of the Wisconsin Package Version 10.1 (Genetics Computer Group, Madision, Wis., USA) using the default scoring matrix with a gap creation penalty of 50 and a gap extension penalty of 3.

It will be clear that whenever nucleotide sequences of RNA molecules are defined by reference to nucleotide sequence of corresponding DNA molecules, the thymine (T) in the nucleotide sequence should be replaced by uracil (U). Whether reference is made to RNA or DNA molecules will be clear from the context of the application.

The functional level of the sucrose synthase type C isoform, or sucrose synthases with similar characteristics may also be increased by in vivo induced sequence variation. To this end, methods for induced sequence variation known in the art as described elsewhere in this document, may be applied to fiber-producing plants, such as cotton plants and fiber producing plants can be identified which exhibit a higher activity for sucrose synthase isoform C activity (e.g. using the enzymatic assay described herein) or which exhibit a higher concentration of sucrose synthase, particularly of sucrose synthase isoform C in the apoplastic fluid or cell wall, particularly during the developmental phase of the secondary cell wall formation, and particularly in cell walls of the fiber or the apoplastic fluid surrounding these fibers.

For some fiber-producing plants, it may be advantageous to modify the fiber characteristics by decreasing the functional level of sucrose synthase isoform C or sucrose synthases with similar characteristics. This may again be conveniently achieved by introduction of a gene encoding a silencing RNA.

In one embodiment, the silencing RNA encoding gene may encode a silencing RNA molecule or an inhibitory RNA molecule, capable of reducing the expression of an endogenous gene encoding a sucrose synthase isoform C to alter fiber characteristics. Such reduction of the expression of a gene encoding a sucrose synthase isoform C should occur preferably through post-transcriptional silencing. However, it will be clear that even when an inhibitory RNA molecule decreases the expression of a SusC gene through post-transcriptional silencing, such an RNA molecule may also exert other functions within a cell, such as guiding DNA methylation of the endogenous SusC gene, again ultimately leading to decreased expression of that gene. Also, expression of endogenous SusC genes may be reduced by transcriptional silencing, e.g. by using RNAi or dsRNA targeted against the promoter region of the endogenous SusC gene.

Several methods are available in the art to produce a silencing RNA molecule, i.e. an RNA molecule which when expressed reduces the expression of a particular gene or group of genes, including the so-called “sense” or “antisense” RNA technologies.

Thus in one embodiment, the inhibitory RNA molecule encoding chimeric gene is based on the so-called antisense technology. In other words, the coding region of the chimeric gene comprises a nucleotide sequence of at least 20 consecutive nucleotides of the complement of the nucleotide sequence of an endogenous SusC gene. Such a chimeric gene may be constructed by operably linking a DNA fragment comprising at least 20 nucleotides from a SusC gene, which can be isolated or identified as described elsewhere in this application, in inverse orientation to a plant expressible promoter and 3′ end formation region involved in transcription termination and polyadenylation. It will be clear that there is no need to know the exact nucleotide sequence or the complete nucleotide sequence of such a DNA fragment from the isolated SusC gene.

In another embodiment, the inhibitory RNA molecule encoding chimeric gene is based on the so-called co-suppression technology. In other words, the coding region of the chimeric gene comprises a nucleotide sequence of at least 20 consecutive nucleotides of the nucleotide sequence of an endogenous SusC gene of the plant. Such a chimeric gene may be constructed by operably linking a DNA fragment comprising at least 20 nucleotides from a SusC gene, which may be isolated or identified as described elsewhere in this application, in direct orientation to a plant expressible promoter and 3′ end formation region involved in transcription termination and polyadenylation. Again, it will be clear that there is no need to know the exact nucleotide sequence or the complete nucleotide sequence of such a DNA fragment from the isolated SusC gene.

The efficiency of the above mentioned chimeric genes in reducing the expression of the endogenous SusC genes may be further enhanced by the inclusion of DNA element which result in the expression of aberrant, unpolyadenylated inhibitory RNA molecules or results in the retention of the inhibitory RNA molecules in the nucleus of the cells. One such DNA element suitable for that purpose is a DNA region encoding a self-splicing ribozyme, as described in WO 00/01133 (incorporated by reference). Another such DNA element suitable for that purpose is a DNA region encoding an RNA nuclear localization or retention signal, as described in PCT/AU03/00292 published as WO03/076619 (incorporated by reference).

A convenient and very efficient way of downregulating the expression of a gene of interest uses so-called double-stranded RNA (dsRNA) or interfering RNA (RNAi), as described e.g. in WO99/53050 (incorporated by reference). In this technology, an RNA molecule is introduced into a plant cell, whereby the RNA molecule is capable of forming a double stranded RNA region over at least about 19 to about 21 nucleotides, and whereby one of the strands of this double stranded RNA region is about identical in nucleotide sequence to the target gene (“sense region”), whereas the other strand is about identical in nucleotide sequence to the complement of the target gene or of the sense region (“antisense region”). It is expected that for silencing of the target gene expression, the nucleotide sequence of the 19 consecutive nucleotide sequences may have one mismatch, or the sense and antisense region may differ in one nucleotide. To achieve the construction of such RNA molecules or the encoding chimeric genes, use can be made of the vector as described in WO 02/059294.

Thus, in one embodiment of the invention, a method for altering fiber characteristics of a fiber producing plant, such as cotton, is provided comprising the step of introducing a chimeric gene into a cell of the fiber producing plant, wherein the chimeric gene comprises the following operably linked DNA elements:
(a) a plant expressible promoter, preferably a plant expressible promoter which controls transcription preferentially in the fiber cells;
(b) a transcribed DNA region, which when transcribed yields a double-stranded RNA molecule capable of reducing the expression of a SusC gene endogenous to the fiber producing plant, and the RNA molecule comprising a first and second RNA region wherein
   i) the first RNA region comprises a nucleotide sequence of at least 19 consecutive nucleotides having at least about 94% sequence identity to the nucleotide sequence of an endogenous SusC gene;
   ii) the second RNA region comprises a nucleotide sequence complementary to the at least 19 consecutive nucleotides of the first RNA region;
   iii) the first and second RNA region are capable of base-pairing to form a double stranded RNA molecule between at least the 19 consecutive nucleotides of the first and second region; and
(c) a 3′ end region comprising transcription termination and polyadenylation signals functioning in cells of the plant.

The length of the first or second RNA region (sense or antisense region) may vary from about 19 nucleotides (nt) up to a length equaling the length (in nucleotides) of an endogenous SusC gene. The total length of the sense or antisense nucleotide sequence may thus be at least at least 25 nt, or at least about 50 nt, or at least about 100 nt, or at least about 150 nt, or at least about 200 nt, or at least about 500 nt. It is expected that there is no upper limit to the total length of the sense or the antisense nucleotide sequence. However for practical reasons (such as e.g. stability of the chimeric genes) it is expected that the length of the sense or antisense nucleotide sequence should not exceed 5000 nt, particularly should not exceed 2500 nt and could be limited to about 1000 nt.

It will be appreciated that the longer the total length of the sense or antisense region, the less stringent the requirements for sequence identity between these regions and the corresponding sequence in an endogenous SusC gene or its complement. Preferably, the nucleic acid of interest should have a sequence identity of at least about 75% with the corresponding target sequence, particularly at least about 80%, more particularly at least about 85%, quite particularly about 90%, especially about 95%, more especially about 100%, quite especially be identical to the corresponding part of the target sequence or its complement. However, it is preferred that the nucleic acid of interest always includes a sequence of about 19 consecutive nucleotides, particularly about 25 nt, more particularly about 50 nt, especially about 100 nt, quite especially about 150 nt with 100% sequence identity to the corresponding part of the target nucleic acid. Preferably, for calculating the sequence identity and designing the corresponding sense or antisense sequence, the number of gaps should be minimized, particularly for the shorter sense sequences.

dsRNA encoding chimeric genes according to the invention may comprise an intron, such as a heterologous intron, located e.g. in the spacer sequence between the sense and antisense RNA regions in accordance with the disclosure of WO 99/53050 (incorporated herein by reference).

In another embodiment, the silencing RNA or inhibitory RNA molecule may be a microRNA molecule, designed, synthesized and/or modulated to target and cause the cleavage of endogenous SusC genes in a fiber producing plants, such as a cotton plant. Various methods have been described to generate and use miRNAs for a specific target gene (including but not limited to Schwab et al. (2006, Plant Cell, 18(5):1121-1133), WO2006/044322, WO2005/047505, EP 06009836, incorporated by reference). Usually, an existing miRNA scaffold is modified in the target gene recognizing portion so that the generated miRNA now guides the RISC complex to cleave the RNA molecules transcribed from the target nucleic acid. miRNA scaffolds could be modified or synthesized such that the miRNA now comprises 21 consecutive nucleotides of the nucleotide sequence of a susC encoding nucleotide sequence, such as the sequences represented in the Sequence listing, and allowing mismatches according to the herein below described rules. Particularly suitable sequences from which the 21 consecutive nucleotides may be chosen comprise the nucleotide sequences encoding the SusC specific amino acid sequences as described herein.

Thus, in one embodiment, the invention provides a method for down-regulating the expression of a or increasing the resistance of plants to adverse growing conditions, comprising the steps of
a. Introducing a chimeric gene into cells of said plants, said chimeric gene comprising the following operably linked DNA regions:
   i. A plant expressible promoter;
   ii. A DNA region which upon introduction and transcription in a plant cell is processed into a miRNA, whereby the miRNA is capable of recognizing and guiding the cleavage of the mRNA of an endogenous SusC encoding gene of the plant; and
   iii. optionally, a 3′ DNA region involved in transcription termination and polyadenylation.

The mentioned DNA region processed into a miRNA may comprise a nucleotide sequence which is essentially complementary to a nucleotide sequence of at least 21 consecutive nucleotides of a SusC encoding gene, provided that one or more of following mismatches are allowed:
a. A mismatch between the nucleotide at the 5′ end of the miRNA and the corresponding nucleotide sequence in the RNA molecule;
b. A mismatch between any one of the nucleotides in position 1 to position 9 of the miRNA and the corresponding nucleotide sequence in the RNA molecule;
c. Three mismatches between any one of the nucleotides in position 12 to position 21 of the miRNA and the corresponding nucleotide sequence in the RNA molecule provided that there are no more than two consecutive mismatches.

Lowering the functional level of Sucrose synthase isoform C may be advantageous e.g. in fiber cells which lead to fuzz fiber in cotton to reduce or avoid the divergence of energy and metabolites for the production of the less favored fuzz fiber at the expense of lint production.

As used herein, an “endogenous gene” is a gene that naturally occurs in the species of the fiber-producing plant that has been chosen for modulation of fiber characteristics, or a gene that occurs naturally in a species of another fiber-producing plant but may be introduced into the species of the fiber-producing plant that has been chosen for modulation of fiber characteristics, by conventional breeding techniques.

It will also be clear that the expression of endogenous SusC gene may also be downregulated using chimeric genes as herein described, wherein the DNA encoding the target specific RNA has a nucleotide sequence of at least 20 consecutive nucleotides selected from the nucleotide sequences encoding the amino acid sequences of SEQ ID No. 2, 3, 5, 7, 9, 11 or 13 or their complement, or wherein the target specific RNA has a nucleotide sequence of at least 20 consecutive nucleotides selected from the nucleotide sequences of SEQ ID No. 1, 4, 6, 8, 10, 12 or 14.

As used herein, the term “promoter” denotes any DNA which is recognized and bound (directly or indirectly) by a DNA-dependent RNA-polymerase during initiation of transcription. A promoter includes the transcription initiation site, and binding sites for transcription initiation factors and RNA polymerase, and can comprise various other sites (e.g., enhancers), at which gene expression regulatory proteins may bind.

As used herein, the term “plant-expressible promoter” means a DNA sequence which is capable of controlling (initiating) transcription in a plant cell. The promoter of the chimeric genes described herein may be naturally associated with the coding regions, or it may be a heterologous promoter. The term plant expressible promoter includes any promoter of plant origin, but also any promoter of non-plant origin which is capable of directing transcription in a plant cell, i.e., certain promoters of viral or bacterial origin such as the CaMV35S, the subterranean clover virus promoter No 4 or No 7, or T-DNA gene promoters and the like.

The plant expressible promoter may be constitutive or it may initiate the transcription of the downstream linked region in a spatial or temporary manner. A plant-expressible promoter that controls initiation and maintenance of transcription preferentially in fiber cells is a promoter that drives transcription of the operably linked DNA region to a higher level in fiber cells and the underlying epidermis cells than in other cells or tissues of the plant. Such promoters include the promoter from cotton from a fiber-specific β-tubulin gene (as described in WO0210377), the promoter from cotton from a fiber-specific actin gene (as described in WO0210413), the promoter from a fiber specific lipid transfer protein gene from cotton (as described in U.S. Pat. No. 5,792,933), a promoter from an expansion gene from cotton (WO9830698) or a promoter from a chitinase gene in cotton (US2003106097) or the promoters of the fiber specific genes described in U.S. Pat. No. 6,259,003 or U.S. Pat. No. 6,166,294. The promoter may also be inducible by chemical compound (usually in combination with a transcriptional activator) as described e.g. in WO93/21334, U.S. Pat. No. 5,514,578, EP0823480, WO98/05789, WO01/34821 or WO02/20811.

The methods described herein will change the characteristics of fibers produced by the modified plants including strength, length, fiber fineness, fiber maturity ratio, immature fiber content, fiber uniformity, and micronaire.
The fiber micronaire (which can be determined by HVI) is a unit less measurement that depends both on fiber maturity (or wall thickness determined by secondary wall cellulose content) and fiber diameter.
Fiber bundle strength (by HVI) is expressed in units of (cN/tex). It is the specific strength of the fiber bundle is which the individual fiber fineness (tex) is calculated from the Micronairevalue.
Fiber fineness (by AFIS) is expressed as (mTex). It represents the weight, in milligrams, of one kilometer of the fiber. One thousand meters of fibers with a mass of 1 milligram equals 1 millitex.
The fiber maturity ratio (by AFIS) is an expression of the degree of cell wall thickening (depending on secondary cell wall cellulose deposition). It is the ratio of fibers with a 0.5 (or more) circularity ratio divided by the amount of fibers with 0.25 (or less) circularity. (Fibers with thicker walls are less prone to collapse and remain more circular upon drying.) The higher the maturity ratio, the more mature the fibers are and the better the fibers are for dyeing.
The immature fiber content (“IFC %”, by AFIS) is the percentage of fibers with less than 0.25 maturity. The lower the IFC %, the more suitable the fiber is for dyeing.
Several different units are used as indicators of fiber length. shows values for three of these as now described. Upper half mean (“UHM”, by HVI) is the mean length of the longest one half of the fibers (weight biased).
The fiber Uniformity Index (“UI”, by HVI) expresses the ratio of the mean value (Mean Length) to the Upper Half Mean Length. It is a measure of the fiber length scatter within the population; if all fibers were the same length UI would equal 100%. Short Fiber Content (“SFC %”, by HVI) is the percentage of fibers less than [½]″ long on a weight basis. HVI is thought to measure Short Fiber Content as determined by genetics only since the measurement does not impose additional potential fiber breaking stress.

Other fiber length indicators include the weight basis length (“L(w)” [in], by AFIS) is the average length of fibers calculated on a weight basis. The number basis length (“L(n)” [in], by AFIS) is the mean length of fibers calculated by number. The length “L5% (n)” [in] (by AFIS) is the 5% span length, or the length spanned by 5% of the fibers when they are parallel and randomly distributed. The length “L2.5% (n)” [in] (by AFIS) is the 2.5% span length, or the length spanned by 2.5% of the fibers when they are parallel and randomly distributed. The “UQL (w)” [in] (by AFIS) is the upper quartile length of fibers by weight, or the length exceeded by 25% of the fibers by weight. Finally, the “SFC (n)” [in] and “SFC (w)” [in] (by AFIS) are the percentage of fibers less than 0.50 inches long on a number and weight basis, respectively. In contrast to HVI, AFIS beats the fibers before taking these measurements, which has potential to cause fiber breakage. Therefore, AFIS SFC values are a good indication of the characteristics of the fiber after normal processing

It will be clear that the methods and means described herein to alter the characteristics of fibers in fiber-producing plants such as cotton plants may be combined with other methods to alter fiber characteristics as known in the art.

In one embodiment of the invention, the methods and means of the current application are combined with those described in WO2005/017157 (incorporated herein by reference) or WO01/17333. It is expected that the combined expression of the genes will result in a synergistic effect on the increase of the fiber length and/or strength. The methods and means of the current application can also be combined with other methods and means directed towards modification of the fiber characteristics as described e.g. in PCT/EP2006/005853 or in WO98/00549.

The chimeric genes may be introduced by subsequent transformation into cells of one plant, or may be combined into cells of one plant by crossing between plants comprising one chimeric gene each.

As the onset of secondary cell wall synthesis in fibers, such as cotton fibers overlaps with the fiber elongation phase, it may further be advantageous to delay the onset of expression of the Sucrose C isoform until a later stage, thereby prolonging the fiber elongation phase. This delay in SusC gene expression can still be combined with an increased expression of the SusC gene expression at that later stage. One way to achieve this delay would be to “exchange” the endogenous SusC gene(s) for a SusC gene whose expression is turned on at a later change than the endogenous SusC genes, e.g. by reducing the expression of the endogenous SusC gene, as herein described, and introduction of a SusC coding region under the control of a promoter which is turned on at a later developmental stage of secondary cell wall synthesis, such as e.g. the E6 promoter (John M E, Keller G, Proc Natl Acad Sci USA 1996, 93:12768-12773). The chimeric susC encoding region under control of a promoter expressed at a late stage in secondary cell wall synthesis may be introduced ectopically, or the endogenous SusC gene may be introduced through homologous recombination techniques, as described e.g. in PCT/EP2006/003086. As the susC gene of Gossypium barbadense cultivars, such as PIMA varieties, appears to be expressed at a later stage than the susC gene of G. hirsitum varieties, “exchange” of the susC gene in G. hirsitum varieties by susC genes from G. barbadense cultivars is expected to prolong the fiber elongation phase and lead to longer cotton fibers.

The invention also encompasses the chimeric genes herein described, as well as plants, seeds, tissues comprising these chimeric genes, and fibers produced from such plants

Methods to transform plants are well known in the art. Methods to transform cotton plants are also well known in the art. Agrobacterium-mediated transformation of cotton has been described e.g. in U.S. Pat. No. 5,004,863 or in U.S. Pat. No. 6,483,013 and cotton transformation by particle bombardment is reported e.g. in WO 92/15675.

The chimeric genes according to the invention may be introduced into plants in a stable manner or in a transient manner using methods well known in the art. The chimeric genes may be introduced into plants, or may be generated inside the plant cell as described e.g. in EP 1339859.

The chimeric genes may be introduced by transformation in cotton plants from which embryogenic callus can be derived, such as Coker 312, Coker310, Coker 5Acala SJ-5, GSC25110, FIBERMAX 819, Siokra 1-3, T25, GSA75, Acala SJ2, Acala SJ4, Acala SJ5, Acala SJ-C1, Acala B1644, Acala B1654-26, Acala B1654-43, Acala B3991, Acala GC356, Acala GC510, Acala GAM1, Acala C1, Acala Royale, Acala Maxxa, Acala Prema, Acala B638, Acala B1810, Acala B2724, Acala B4894, Acala B5002, non Acala “picker” Siokra, “stripper” variety FC2017, Coker 315, STONEVILLE 506, STONEVILLE 825, DP50, DP61, DP90, DP77, DES119, McN235, HBX87, HBX191, HBX107, FC 3027, CHEMBRED A1, CHEMBRED A2, CHEMBRED A3, CHEMBRED A4, CHEMBRED B1, CHEMBRED B2, CHEMBRED B3, CHEMBRED C1, CHEMBRED C2, CHEMBRED C3, CHEMBRED C4, PAYMASTER 145, HS26, HS46, SICALA, PIMA S6 ORO BLANCO PIMA, FIBERMAX FM5013, FIBERMAX FM5015, FIBERMAX FM5017, FIBERMAX FM989, FIBERMAX FM832, FIBERMAX FM966, FIBERMAX FM958, FIBERMAX FM989, FIBERMAX FM958, FIBERMAX FM832, FIBERMAX FM991, FIBERMAX FM819, FIBERMAX FM800, FIBERMAX FM960, FIBERMAX FM966, FIBERMAX FM981, FIBERMAX FM5035, FIBERMAX FM5044, FIBERMAX FM5045, FIBERMAX FM5013, FIBERMAX FM5015, FIBERMAX FM5017 or FIBERMAX FM5024 and plants with genotypes derived thereof.

“Cotton” as used herein includes Gossypium hirsutum or Gossypium barbadense. “Cotton progenitor plants” include Gossypium arboretum, Gossypium herbaceum and Gossypium raimondii and Gossypium longicalyx

Nevertheless, the methods and means of the current invention may also be employed for other plant species such as hemp, jute, flax and woody plants, including but not limited to Pinus spp., Populus spp., Picea spp., Eucalyptus spp. etc.

The obtained transformed plant can be used in a conventional breeding scheme to produce more transformed plants with the same characteristics or to introduce the chimeric gene according to the invention in other varieties of the same or related plant species, or in hybrid plants. Seeds obtained from the transformed plants contain the chimeric genes of the invention as a stable genomic insert and are also encompassed by the invention.

In another embodiment, a method is provided for identifying allelic variations of the proteins involved in fiber characteristics in a population of different genotypes or varieties of a particular plant species, preferably a fiber-producing plant species, which are correlated either alone or in combination with the quantity and/or quality of fiber production. This method includes the following steps:
a) providing a population of different varieties or genotypes of a particular plant species or interbreeding plant species comprising different allelic forms of the nucleotide sequences encoding Sucrose synthase isoform C, such as nucleotide sequences encoding SEQ ID No 1, 4, 6, 8, 10, 12 or 14. The different allelic forms may be identified using the methods described elsewhere in this application. Preferably, a segregating population is provided, wherein different combinations of the allelic variations of the SusC proteins are present. Methods to produce segregating populations are well known in the art of plant breeding;
b) determining parameters related to fiber characteristics for each individual of the population;
c) determining the presence of a particular allelic form of the nucleotide sequences encoding SusC for each individual of the population; and
d) correlating the occurrence of particular fiber characteristic with the presence of a particular allelic form of the mentioned nucleotide sequence or a particular combination of such allelic forms.

The resulting information may be used to accelerate breeding program varieties with particular fiber or drought resistance characteristics, by determining the presence or absence of allelic forms, using conventional molecular biology techniques.

Allelic forms of the SusC gene associated with particular fiber characteristics may also be identified, isolated and introduced into plants, such as cotton plants, whereby the expression of the endogenous SusC has been reduced or eliminated. Such reduction of expression of the endogenous SusC genes can be conveniently achieved by posttranscriptional or transcriptional silencing as herein described, or may be achieved by inactivation, such as by deletion, of the endogenous SusC genes. Introduction of the allelic forms may be achieved by breeding techniques, or by transformation with the isolated genes.

In another embodiment of the invention, antibodies raised against the novel type of Sucrose synthase genes are provided, particularly antibodies recognizing the SusC proteins having the amino acid sequences of SEQ ID No.s 2, 3, 5, 7, 9, 11 or 13.

As used herein “comprising” is to be interpreted as specifying the presence of the stated features, integers, steps or components as referred to, but does not preclude the presence or addition of one or more features, integers, steps or components, or groups thereof. Thus, e.g., a nucleic acid or protein comprising a sequence of nucleotides or amino acids, may comprise more nucleotides or amino acids than the actually cited ones, i.e., be embedded in a larger nucleic acid or protein. A chimeric gene comprising a DNA region, which is functionally or structurally defined, may comprise additional DNA regions etc.

The following non-limiting Examples describe the identification of a novel type of sucrose synthase involved in secondary plant cell wall synthesis, and chimeric genes for the alteration of fiber characteristics in fiber producing plants such as cotton and uses thereof. Unless stated otherwise in the Examples, all recombinant DNA techniques are carried out according to standard protocols as described in Sambrook et al. (1989) Molecular Cloning: A Laboratory Manual, Second Edition, Cold Spring Harbor Laboratory Press, NY and in Volumes 1 and 2 of Ausubel et al. (1994) Current Protocols in Molecular Biology, Current Protocols, USA. Standard materials and methods for plant molecular work are described in Plant Molecular Biology Labfax (1993) by R. D. D. Croy, jointly published by BIOS Scientific Publications Ltd (UK) and Blackwell Scientific Publications, UK.

Throughout the description and Examples, reference is made to the following sequences represented in the sequence listing:
SEQ ID No.: 1: Nucleotide sequence of cDNA encoding sucrose synthase type C from cotton.
SEQ ID No.: 2: Amino acid sequence of cDNA encoded sucrose synthase type C from cotton.
SEQ ID No.: 3: Amino acid sequence of SusC from a Gossypium hirsutum cv.
SEQ ID No.: 4: Nucleotide sequence of SusC genomic DNA from Gossypium hirsutum cv.
SEQ ID No.: 5: Amino acid sequence of SusC from a Gossypium barbadense cv.
SEQ ID No.: 6: Nucleotide sequence of SusC genomic DNA from Gossypium barbadense cv. encoding the SUS C protein of SEQ ID No.: 7.
SEQ ID No.: 7: Amino acid sequence of SusC from Gossypium arboreum.
SEQ ID No.: 8: Nucleotide sequence of SusC genomic DNA from Gossypium arboretum.
SEQ ID No.: 9: Amino acid sequence of SusC from Gossypium raimondii.
SEQ ID No.: 10: Nucleotide sequence of SusC genomic DNA from Gossypium raimondii.
SEQ ID No.: 11: Amino acid sequence of SusC from Gossypium longicalyx subtype 1
SEQ ID No.: 12: Nucleotide sequence of SusC genomic DNA from Gossypium longicalyx subtype 1
SEQ ID No.: 13: Amino acid sequence of SusC from Gossypium longicalyx subtype 2
SEQ ID No.: 14: Nucleotide sequence of SusC genomic DNA from Gossypium longicalyx subtype 2
SEQ ID No.: 15: Amino acid sequence of the C-terminal consensus sequence of SusC
SEQ ID No.: 16: Amino acid sequence of the N-terminal consensus sequence of SusC
SEQ ID No.: 17: oligonucleotide sequence used as SUS C specific primer (5′UTR)
SEQ ID No.: 18: oligonucleotide sequence used as SUS C specific primer (3′UTR)

### EXAMPLES

### Example 1

### Identification of Sucrose Synthases of the C-Type in Gossypium Species

Identification of SusC. It has been shown that there are at least 3 classes of Sus genes expressed in cotton fiber during development, termed the A, B and C-types. The A/D-type is homologous to the sequence previously reported (Accession number U73588) and the B-type shows strong sequence similarity to the published sequence. The C-type sequences provided in this document are novel and show differential expression between elongation and secondary cell wall phase. The C-type appears to be the isoform appearing at later stages in fibre development. SusC is only 76% identical to the A and B-type proteins at the amino acid level. Using RTPCR and Northern analysis it has been shown that the developmental expression profile of the C-type transcript precedes secondary cell wall synthesis but shows a pattern more commensurate with a role in this phase of fiber development while the A and B-types are expressed highly early in fibre elongation. The C-type gene is also expressed strongly in stem tissue.

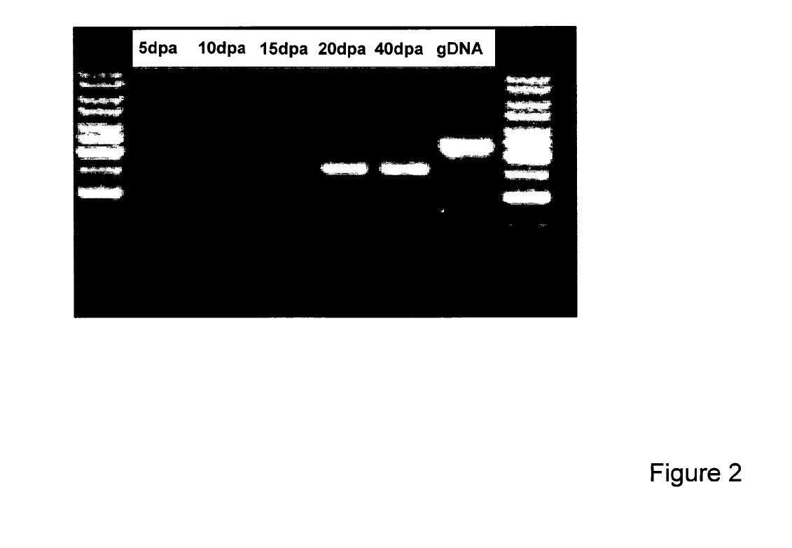

cDNA was isolated from fiber tissue at different stages of its development: 5, 10, 15, 20, 40 days post anthesis (dpa). Libraries were made from these cDNA s and DNA was isolated. This DNA was then used to characterize the susyC expression using 9 ng from each library DNA as starting material for a PCR reaction using as primers the SusC type specific primer of SEQ ID Nos: 17 and 18. The expected size of the amplified fragment is 2550 by for the cDNA and 3353 by for genomic DNA. Appropriate controls were run to validate the semiquantitative character of this assay. FIG. 2 shows the results, and allows to conclude that the SusC mRNA is relatively more abundant at 20-40 dpa.

Protein fractionation. Protein extracts from fibres at elongation stage (8-11 DAF) and secondary cell wall synthesis stage (15-25 DAF) were fractionated to provide soluble (cytosolic) and microsomal (plasma membrane, cytoskeleton and tonoplast enriched) fractions. The microsomal fraction was further purified to enrich for plasma membrane proteins. This PM fraction no longer has any cytoskeletal or cell wall contamination, as judged by western blotting against actin. Also included was another cellular fraction comprising apoplastic proteins from intact fibers obtained by gentle washing with isotonic buffer including protease inhibitors and EGTA. These protein preps were run on SDS-PAGE and the Sus proteins identified by western blotting. The gel fragments were excised and subjected to tryptic digest followed by tandem mass-spec to identify the form of Sus present. Examples of Signature peptides used to identify the SusC isoform are shown in Table 1A and 1B. The predicted molecular weight of Sus-C is 90.3 kDa as opposed to 92.4 for the A/D and B-type proteins permitting resolution of the C form from the A/B isoforms on SDS-PAGE. This resolution was confirmed by analysis of these 2 bands by peptide MS-MS (the larger band contains signature peptides from A and B while the smaller band alone has unique C-type peptides).

TABLE 1A
SusC specific peptides detected in apoplast

| | | |
|---|---|---|
| 8d apoplast | 17d apoplast | 25d apoplast |

| | | |
|---|---|---|
| No C-type detected | 9.20e + 005 | 1.45e + 006 |
| | (R)KAEEYLTPLSSDTPYSVFEK( ) | (R)IQDVNSLQHARL( ) |
| | 8.17e + 005 | 2.37e006 |
| | (K)LLTLTGVYGFSK(H) | (R) KAEEYLTPLS SDTPYSVFEK( ) |
| | 1.32e + 006 | 2.16e006 |
| | (K)YTWQIYSEK( ) | (K)NLTGLVEFYAK(N) |
| | | 6.90e + 005 |
| | | (R)STQEAVVSSPLVALAIR( ) |
| | | 7.45e + 006 |
| | | (K)LQGLDITPR(I) |
| | | 3.64e + 006 |
| | | (K)YTWQIYSEK( ) |
| | | 5.53e + 006 |
| | | (R)VVDGIDVFDPK(F) |
| | | 3.34e + 006 |
| | | (K) LLTLTGVYGFSK(H) |
| | | 4.17e + 005 |
| | | (K)D TLGQYESHIAFTLPGLYR(V) |
| | | 3.50e + 005 |
| | | (K)LFVEEMPVAEYLR(L) |

TABLE 1B
SusC specific peptides detected in microsomal fraction

| | | | |
|---|---|---|---|
| 8d microsomal | 13d mircosomal | 20d microsomal | 25d microsomal |

| | | | |
|---|---|---|---|
| No C-type detected | No C-type detected | 6.06E + 006 | 3.94E + 006 |
| | | (R)KAEEYLTPLSSDTPYSVFEK( ) | (R) KAEEYLTPLS SDTPYSVFEK( ) |
| | | 7.64E + 006 | 1.13E + 006 |
| | | (K)LLTLTGVYGF SK(H) | (K)LFVEE MPVAEYLR(L) |
| | | 2.31E + 006 | 3.89E + 006 |
| | | (R)IQ DVNSLQHALR( ) | (R)VVDGIDVFDPK(F) |
| | | 6.70E + 006 | 3.68E + 006 |
| | | (R)VVDGIDVFDPK(F | (K)LLTLTGVYGF SK(H) |
| | | 1.82E + 006 | 3.97E + 005 |
| | | (R)YIEmLHAWmYNNR(V) | (K)SGFNIDPYNGDLAAETLANFFEK(C) |
| | | 3.19E + 006 | 2.63E + 006 |
| | | (K)YTWQIYSEK( ) | (K)NLTGLV EFYAK(N) |
| | | 1.99E + 006 | 1.26E + 006 |
| | | (R)STQEAVVSSPLVALAIR( ) | (R)STQEAVV SSPLVALAIR( |
| | | 1.05E + 006 | 2.05E + 006 |
| | | (R)LGESLATHPQQAK(S) | (K)NLTGLVEFYAK(N) |
| | | 5.72E + 006 | 3.01E + 006 |
| | | (K)LFVEEmPVAEYLR(L) | (K)D TLGQYESHIA FTLPGLYR(V) |
| | | 8.25E + 005 | 2.18E + 006 |
| | | (K)DTLGQYESHIAFTLPGLYR(V) | (R)IQ DVNSLQHALR( ) |
| | | 5.25E + 006 | 8.02E + 005 |
| | | (K)LQGLDITPR(I) | (R)YIEmLH AWmYNNR(V) |
| | | 3.28E + 006 | 3.60E + 005 |
| | | (K)NLTGLVEFYAK(N) | (K)TKYPDSDIN WK(Q |
| | | | 9.91E + 005 |
| | | | (K)SIGNGmDFLNR(H) |
| | | | 5.35E + 005 |
| | | | (R)L GESLATHPQQ AK(S) |
| | | | 5.59E + 005 |
| | | | (R)ALEEELLHR(F) |
| | | | 2.77E + 006 |
| | | | (K)LFVEE mPVAEYLR(L) |

Table 2 presents a summary of the mass-spec and SDSPAGE/Western data. Of particular note is the low abundance of Sus-C protein from soluble, microsomal and PM fractions. This seems paradoxical considering the high levels of transcript present at the later stages of fibre development. Apoplastic washes at the later stage of development reveal that the C-type protein is present at high levels (relative to A) on western blots, the converse of the observations for the cytosolic, microsomal and PM fractions. Sus-C is highly abundant at 25 DAF and could not be detected at 8 DAF by peptide MS-MS. In contrast, Sus-A and possibly Sus-B are ubiquitous during development (in all fractions). Sus-A is the major, and possibly the only isoform in PM fractions and runs as a 85 kDa protein on SDS-PAGE

TABLE 2
Summary of the mass-spec and SDSPAGE/Western data

| | | |
|---|---|---|
| 8 | dpa microsomal (upper band) | Susy A + B |
| 8 | dpa microsomal (lower band) | Susy A or A + B |
| 15 | dpa microsomal | Susy A + C or A + B + C |
| 8 | dpa plasma membrane | Susy A or A + B |
| 15 | dpa plasma membrane | Susy A or A + B |
| 8 | dpa soluble | Susy A or A + B |
| 15 | dpa soluble | Susy A + B |
| 8 | dpa apoplast (upper band) | Susy A + B (very low amount) |
| 8 | dpa apoplast (lower band) | Susy A + B (predominant band) |
| 25 | dpa apoplast (upper band) | Susy A + B + C *(very low amount) |
| 25 | dpa apoplast (lower band) | Susy C (predominant band) |

Presence of C in this sample is most likely due to contamination from large lower band which corresponds to SusC.

The number of peptide matches for Sus-C in the apoplast fraction at 25 DAF and the western signal indicate that this isoform is abundant, and only present predominantly in the apoplast at 25 DAF. Interestingly, SusA and B are present as the dominant forms in the apoplast in 8 DAF fiber. These results imply a major role for Sus in the cell wall of cotton fiber and the correlation of Sus-C expression at the RNA level with secondary wall formation and the abundance of the protein in the walls at 25 DAF suggests that Sus-C is the major cell wall isoform at secondary wall synthesis stage. EM immunogold work clearly show Sus signal in the outer secondary cell wall. This is entirely consistent with the presence of the Sus-C protein in the apoplast at secondary cell wall stage.

### Example 2

### Analysis of the SusC Isoform and Identification of Differences Between the Different Isoforms of Sucrose Synthase in Cotton and Related Species

Phosphorylation sites: One serine phorphorylation site situated at the N-end of the protein has been well characterized in the literature, having RXXS as a consensus sequence. The phosphorylation of this specific serine amino acid in Zea mays (with the following positions: ¹²RXXS¹⁵) seems to correlate with the release of the susy enzyme from the plasma membrane (Winter H et al., 1997—FEBS Letters 420, 151-155; Hardin S C et al., 2004, Plant Physiology 134, 1427-1438). Mutation of this same serine amino acid at position ⁸RXXS¹¹ causes the mung bean sucrose synthase to be more efficient in using sucrose to make UDPglucose (=higher Km) (Nakai T. et al., 1999, Proc. Natl. Acad. Sci. USA 96, 14-18). Phosphorylation at this specific site has also been characterized in soybean and seems to correlate with the loss in hydrophobicity and thus putatively with the weakening of the bond with the plasma membrane (Zhang X. Q. et al., 1999 Archives of Biochemistry and Biophysics 371, 70-82). This phosphorylation site has also been characterized in other plant species: (sycamore) (Pozueta-Romero J. et al.—2004—Physologia Plantarum 122, p. 275), Japanese pear (Tanase K. et al.—2002—Physologia Plantarum 114, p. 21) and Gossypium hirsutum (upland cotton) (Haigler C. H. et al.—2001—Plant Molecular Biology 47, 29-51). Conclusions from these studies indicate that phosphorylation has an influence on the Km value of the enzyme.

A similar consensus sequence can be identified in all isolated sucrose synthase C proteins with the following amino acid positions: ²⁷RXXS³⁰. This phosphorylation site is thus situated a further inside the protein. A similar signature can be found in other known plant sucrose synthases genes: L19762, AY205302, AY205085, AJ537575, M18745, U2487, X75332, Y76091, X82504, AJ131999. Most of these genes have both phosphorylation sites: ⁸RXXS¹¹ and ²⁷RXXS³⁰. Proteins L19762 and AJ131999 only have the latter.

Another serine phosphorylation site has been characterized in literature: Ser170 from sus1 (maize). This site has been correlated with susy-protein tagging for proteolysis (Hardin S. C. et al., 2003, The Plant journal 35, 588-603). This Ser amino acid is also present in SusC. Only two sucrose synthases do not contain this Serine residue: Atsus5 and AB018561.

Protein domains, functional sites and specific motifs present in SusC (using Inter Pro Scan program). SusC contains two domains called respectively sucrose synthase (from amino acid 1 to 545) and glycosyl transferase (from amino acid 549 to 737) and one family region called sucrose synthase (from amino acid 18 to 794)

UDP-binding site: the following sequence represents the Uridine-binding region on the sucrose synthase C proteins: ²⁷⁰VVIMTPHGYFAQDNVLGYPDTGGQVVYILDQVRALEEELLHRFKLQGLDITPR ILVITRL³²⁹ This sequence shows a very high homology between all the known plant sucrose synthases.

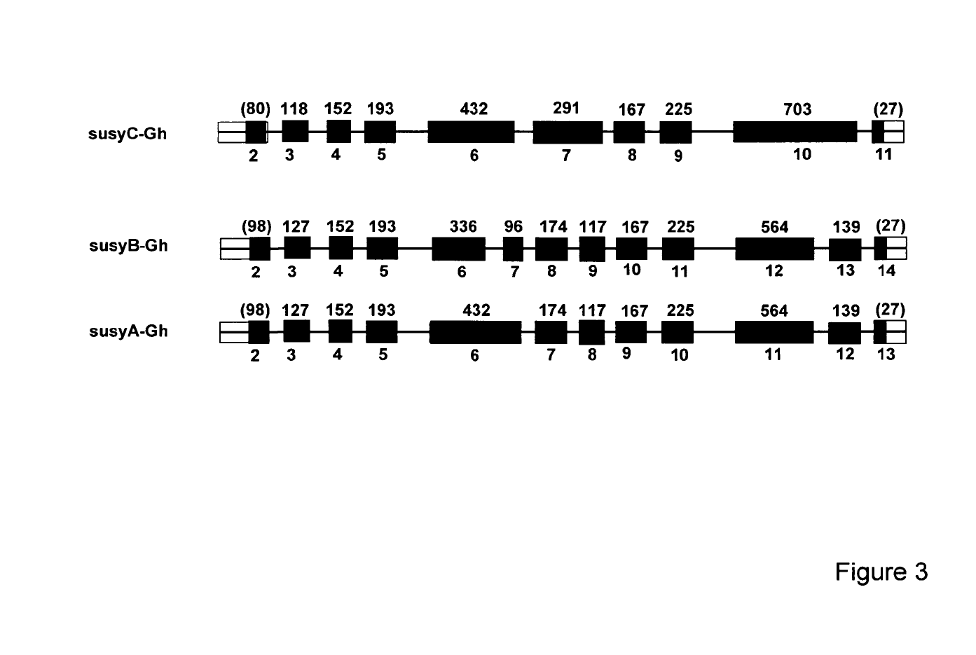

Genomic structure. A comparison was done at the genomic level of the susyC gene with the other isolated cotton susy genes (FIG. 3). It will be immediately clear that the susyC gene has a particular genomic structure in that it has less introns compared to the other susy genes from Gossypium spp. The intron exon structure of the SusyC gene is also considerably different from the Arabidopsis susy genes

Hydrophobicity. The susyC hydrophobicity plot was analyzed by comparing it to other known plant sucrose synthases. The N-terminal end appears to be hydrophobic, a particularity that is not to be found in most of the plant sucrose synthases. The following sucrose synthases were most similar to SusC at this level: SUS6 (Arabidopsis thaliana) sucrose synthase from mung bean (D10266), sucrose synthase from eucalyptus (DD014303) and sucrose synthase from poplar (AY341026).

Actin-binding region. A putative actin-binding region has been described in literature and seems to be present in the sucrose synthases (Winter H. et al.—1998—FEBS letters 430, 205-208; Winter H. and Huber S. C.—2000—Critical Reviews in Plant Sciences 19(1), 31-67). This region binds specifically to F-actin. The putative susyC actin-binding site has the following amino-acid sequence: ³⁸³KDVAAEITKEFQ³⁹⁴. The amino acids highlighted in bold are the ones that are specific for SusyC. The consensus sequence for this site in between the different plant sucrose synthases is: [K*,E,D]-D-[V,A]-[A,G,S,T]-X-E-[L,V,I]-[T,S,A,M,]-[K,R,G,M,L]-E-[F*,L,M]-[Q,N]

Interestingly ³⁸³K(=lys) has a basic side chain while the alternative amino acid residues (E(=glu) and D(=asp)) have acidic side chains. As K is unique for the susyC this may indicate that this specific protein does not bind to the F-actin filaments. Moreover the F(=Phe) amino acid, unique for susyC, is more voluminous than its counterparts L and M from the other plant sucrose synthases. This could further hamper binding with actin.

Amino acid sequences specific for SusC proteins. Upon alignment of the different plant sucrose synthases it became apparent that some regions of the susyC gene were very different and specific. These regions are situated respectively at the N- and C-terminal ends of the susyC gene and have the following amino acid sequence:

A (N-terminal region) = ³⁶HKSQKLLSVLDKEAGNQALDGMV
V⁵⁹
B (C-terminal region) = ⁷⁶⁵AYQEQRGRKRYIEMLHAWMYNNR
VKT⁷⁹⁰

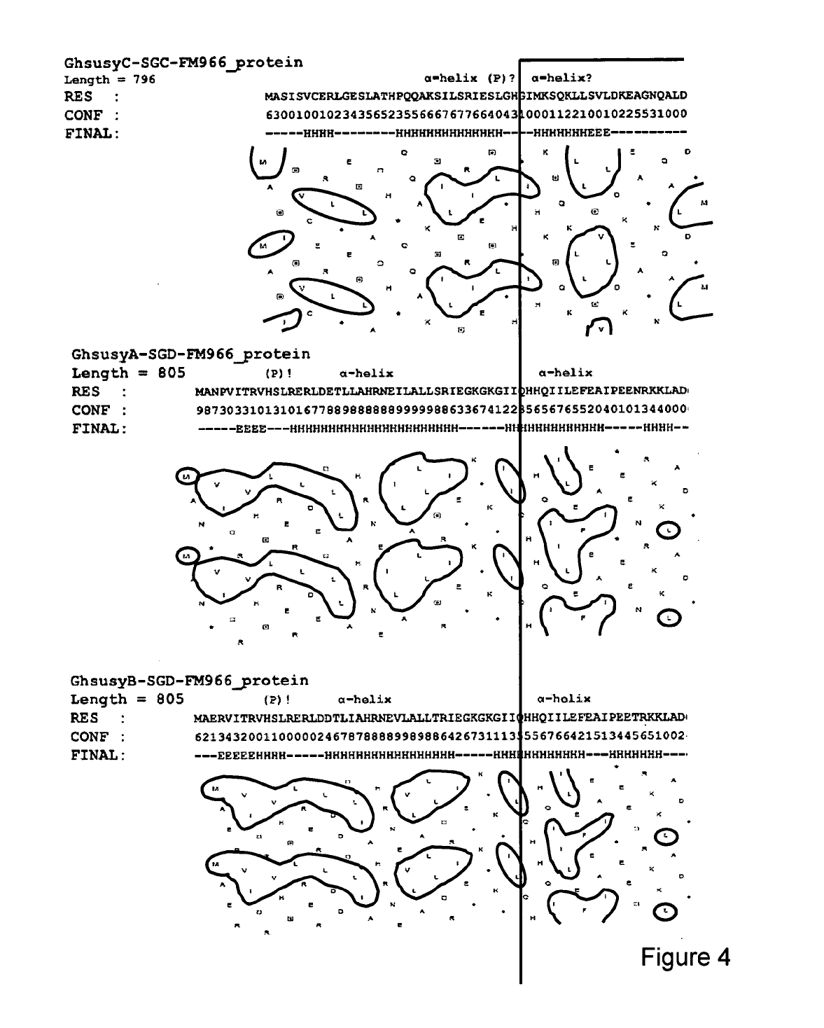

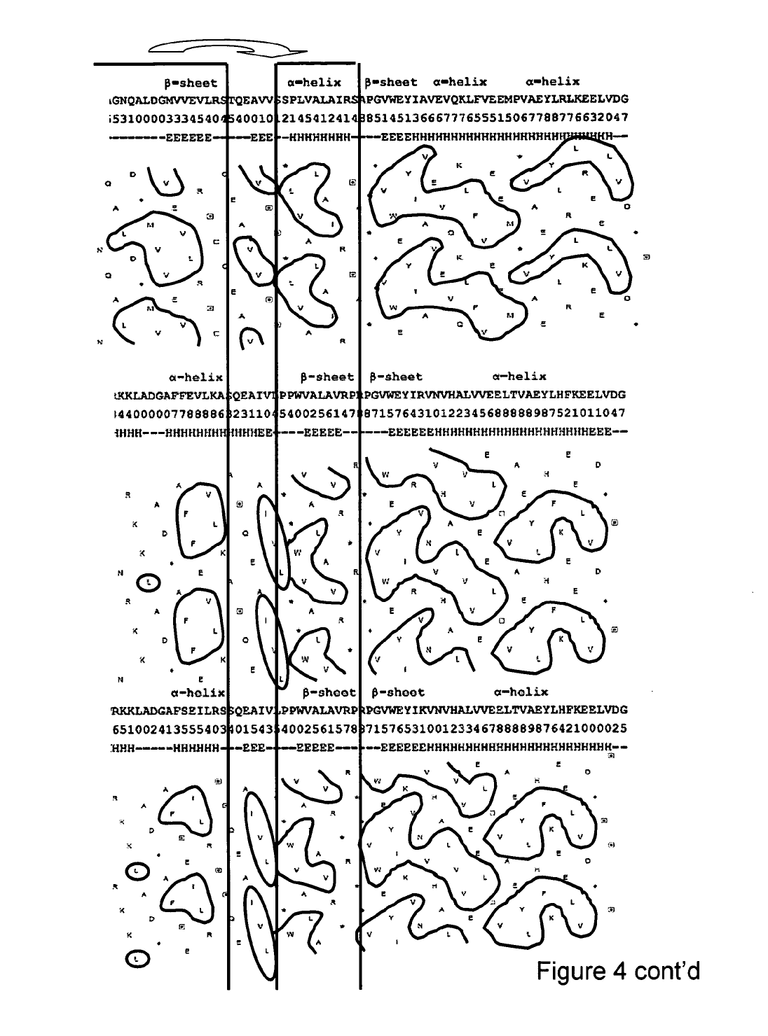

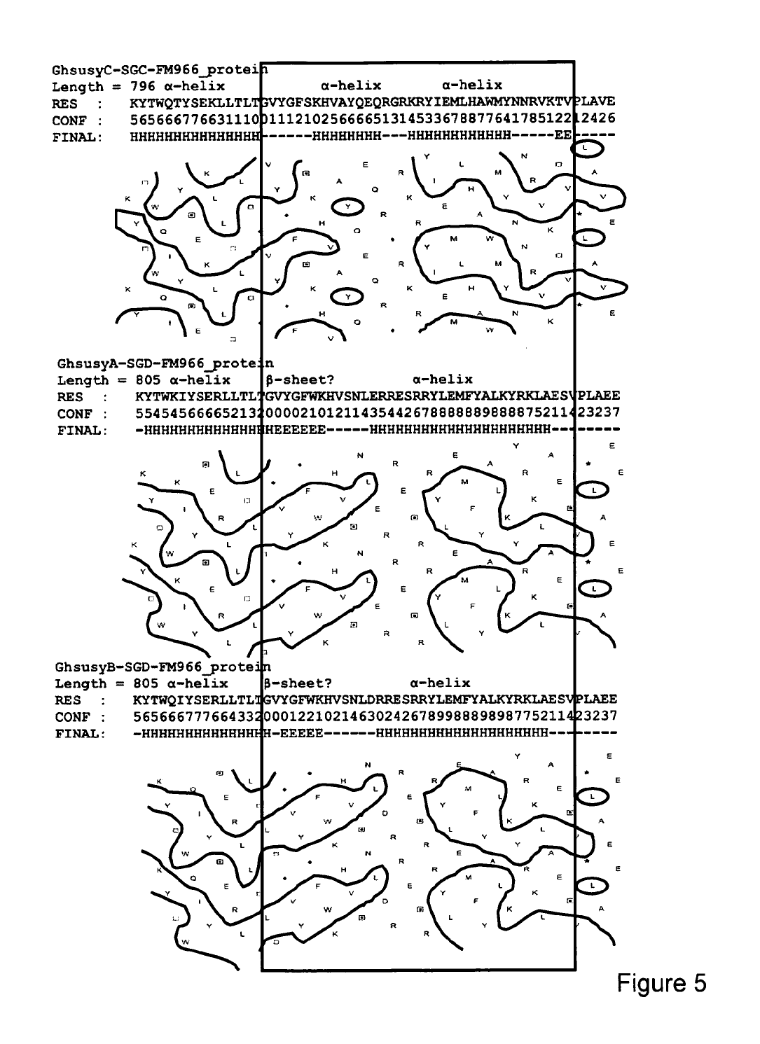

The N-terminal region was analyzed by looking at its predicted 2D structure. For this purpose the following programs were used: HCA (hydrophobic cluster analysis) and JPRED. The results from both programs were aligned and compared with the other Sucrose synthases from Gossypium hirsutum (FIG. 4). It is clear from this analysis that susC at this location is quite unique in structure. The different phosphorylation sites are also indicated confirming the unique feature of the susyC gene. A Similar analysis was performed on the C-terminal region (FIG. 5). Again, the structure of the SusC was different from the structure of the other sucrose synthases from cotton in this region.

The N-terminal and C-terminal SusC specific regions were used to identify EST sequences in databases. The results are summarized in the following table 3

TABLE
number of EST's extracted from some specific tissues and plant species.

| | | primary cell | secondary cell | other | other | |
|---|---|---|---|---|---|---|
| | Ovules | wall | wall | gossypium | plant | |
| | (−3 to 0) | (0 to 15 dpa) | (20 to40 dpa) | species | species | total |
| N-terminal | 0 | 0 | 35 | 1 (=7-10 dpa) | 0 | 36 |
| region | | | | | | |
| C-terminal | 1 | 0 | 30 | 1 (=7-10 dpa) | 0 | 32 |
| region | | | | | | |

### Example 3

### Overexpression of SusC in Cotton

Using standard recombinant DNA techniques, the following DNA elements are operably linked:
a cotton fiber specific promoter as described in WO2004/066571
a DNA region encoding a sucrose synthase C having the nucleotide sequence of SEQ ID No. 1
a 3′ nos terminator region.

This chimeric gene is introduced into a T-DNA vector together with a selectable bar gene. The T-DNA vector is introduced into Agrobacterium tumefaciens and used to produce transgenic cotton plants as described in U.S. Pat. No. 6,483,013.

Transgenic cotton plants comprising the chimeric gene are analyzed for increased expression of SusC, particularly in the fibers, and the fibers obtained from these plants are analyzed for fiber strength, fiber length, fiber maturity ratio, immature fiber content, fiber uniformity and micronaire.

### Example 4

### Downregulation of the Expression of the SusC Isoform in Cotton

Using standard recombinant DNA techniques, the following DNA elements are operably linked:
A CaMV 35S promoter region
a sense RNA encoding region corresponding to the nucleotide sequence of SEQ ID No 1 encoding the N-terminal SusC specific sequence of SEQ ID No 18 or the C-terminal SusC specific sequence of SEQ ID NO 17.
a antisense RNA encoding region corresponding to the complement of the nucleotide sequence of the sense RNA region.
A 3′ nos terminator region

This chimeric gene is introduced into a T-DNA vector together with a selectable bar gene. The T-DNA vector is introduced into Agrobacterium tumefaciens and used to produce transgenic cotton plants as described in U.S. Pat. No. 6,483,013.

Transgenic cotton plants comprising the chimeric gene are analyzed for decreased expression of SusC, particularly in the fibers, and the fibers obtained from these plants are analyzed for fiber strength, fiber length, fiber maturity ratio, immature fiber content, fiber uniformity and micronaire.

### Example 5

### Overexpression of the SusC Isoform in Transgenic Arabidopsis thaliana

Using standard recombinant DNA techniques, the following DNA elements have been operably linked:
A CaMV35S promoter region
The DNA region encoding the SusC of SEQ ID No 2 (SEQ ID No 1 from nt 84 to nt 2474)
A 3′ nos terminator region

In addition, the following DNA element were operably linked
The stalk-specific promoter of the alfalfa S2A gene (Abrahams et al., 1995, Plant. Mol. Biol. 27: 413-528) which results in vascular and trichome preferentional expression in a range of plant species.
The DNA region encoding the SusC of SEQ ID No 2 (SEQ ID No 1 from nt 84 to nt 2474).
A 3′ nos terminator region.

These chimeric genes were introduced into a T-DNA vector together with a selectable bar gene. The T-DNA vector was introduced into Agrobacterium tumefaciens and used to produce transgenic A. thaliana using a floral dip method. Plants have been regenerated and phenotypes recorded at the T2 generation with several interesting phenotypes observed routinely in transgenic plants obtaining one of the above described transgenes.

Many of the transgenic lines for both constructs show enhanced root branching in culture, including extensive lateral shoot growth. Additionally observed were loss of apical dominance, multiple floral bolts, loss of rosette symmetry and compactness, disruption of leaf and floral phyllotaxy and delayed time to flowering.

It is thought that the increased branching and loss of apical dominance is due to reduced cell elongation in meristematic zones due to premature secondary thickening of cell walls.

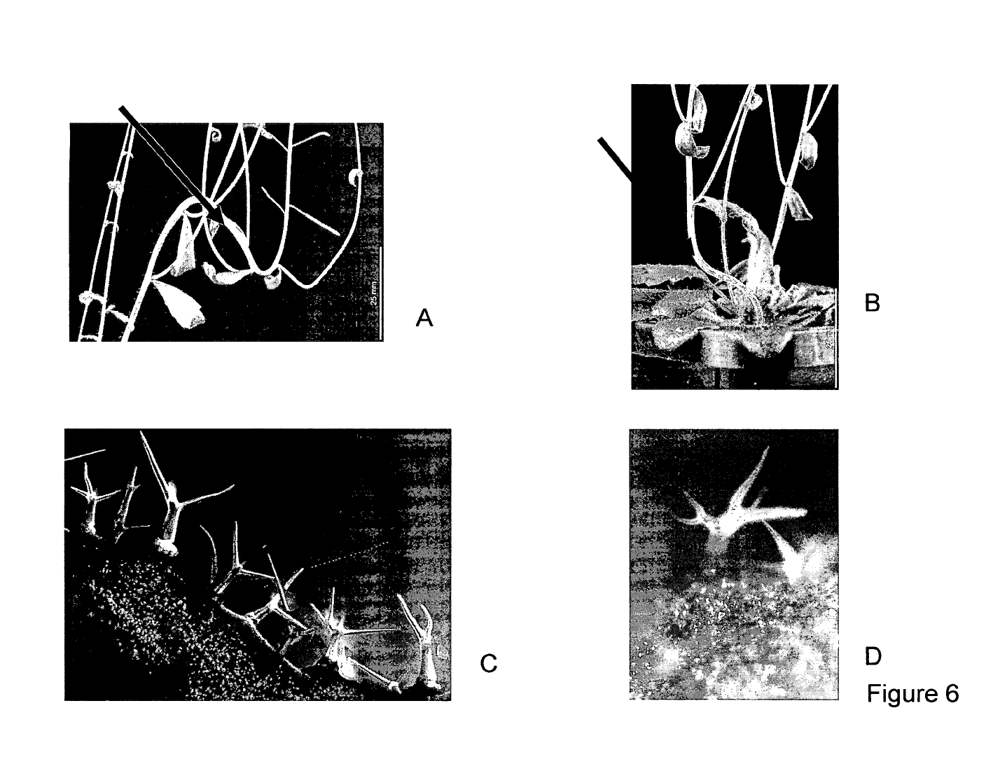

A large proportion of transgenic lines with either of the chimeric genes showed a phenotypic effect on trichome structures. Increased branching and thick pedestals are commonly seen (FIGS. 6 C and D) and trichome density appears to be increased.

Stems and floral bolts of Sus-C transgenic lines commonly show extreme thickening and are bifurcated, possibly fasciated (FIGS. 6 A and B)

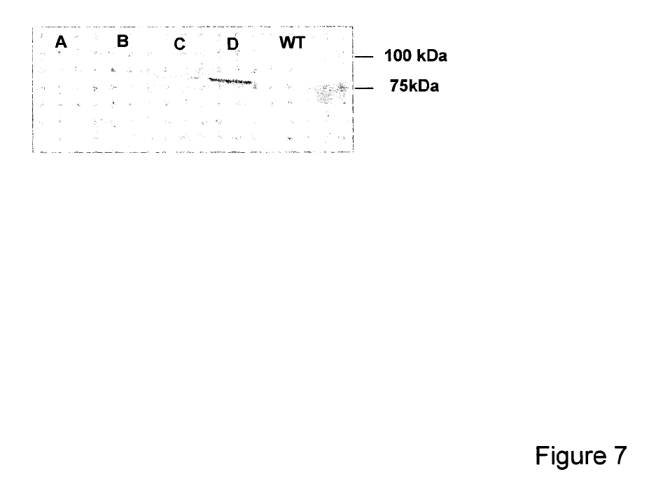

Three methods have been used to correlate phenotype with transgene expression:
1) RT-PCR for Sus-C and the endogenous Sus gene
2) western blotting using the Sus-C specific antibody (see FIG. 7)
3) assay of Sus activity
   Transgenic lines showing high levels of 35S-Sus-C expression, correlating with the most severe phenotypes

1. An isolated sucrose synthase protein comprising
(a) an amino acid sequence comprising at least 80% sequence homology to the amino acid sequence of any one of SEQ ID Nos.: 2, 3, 5, 7, 9, 11 or 13;
(b) an amino acid sequence comprising the amino acid sequence of any one of SEQ ID Nos.: 2, 3, 5, 7, 9, 11 or 13;
(c) an amino acid sequence located in the amino-terminal part of said protein, said amino acid sequence comprising at least about 60% sequence identity to the amino acid sequence of SEQ ID No. 16; or
(d) an amino acid sequence located at the carboxy-terminal part of said protein, said amino acid sequence comprising at least about 60% sequence identity to the amino acid sequence of SEQ ID No. 15.

2. The isolated sucrose synthase protein of claim 1, wherein said protein comprises a hydrophobic N-terminal sequence.

3. The isolated sucrose synthase protein of claim 1, wherein said protein further comprises
(a) the amino acid sequence of SEQ ID No.: 3 from amino acid 383 to amino acid 394;
(b) the amino acid sequence of SEQ ID No.: 3 from amino acid 270 to amino acid 329;
(c) the amino acid sequence of SEQ ID No.: 3 from amino acid 549 to amino acid 737;
(d) the amino acid sequence of SEQ ID No.: 3 from amino acid 1 to amino acid 545; or
(e) the amino acid sequence of SEQ ID No.: 3 from amino acid 18 to amino acid 794.

4. An antibody recognizing the isolated protein of claim 1.

5. An isolated DNA molecule or nucleic acid encoding the sucrose synthase protein of claim 1.

6. The isolated DNA molecule or nucleic acid of claim 5, comprising the nucleotide sequence of any one of SEQ ID Nos.: 1, 4, 6, 8, 10, 12 or 14.

7. An expression cassette comprising the following operably linked DNA molecules a plant expressible promoter; a DNA encoding a sucrose synthase according to claim 1, and, optionally, a transcription termination and polyadenylation region.

8. The expression cassette of claim 7, wherein the plant expressible promoter controls transcription in fiber cells of a fiber producing plant.

9. An expression cassette comprising the following operably linked DNA molecules:
(a) a plant expressible promoter;
(b) a DNA which when transcribed results in an RNA molecule said RNA molecule comprising a nucleotide sequence selected from:
(i) a nucleotide sequence of at least 19 consecutive nucleotides comprising at least about 94% sequence identity to the nucleotide sequence of an endogenous sucrose synthase isoform C encoding gene or to the complement of said nucleotide sequence of an endogenous sucrose synthase isoform C encoding gene;
(ii) a nucleotide sequence of at least 19 consecutive nucleotides comprising at least about 94% sequence identity to a nucleotide sequence according to claim 5 or the nucleotide sequence of any one of SEQ ID Nos.: 1, 4, 6, 8, 10, 12 or 14; and
(iii) optionally, a transcription termination and polyadenylation region.

10. An expression cassette comprising the following operably linked DNA molecules:
(a) a plant expressible promoter;
(b) a DNA, which when transcribed yields a double-stranded RNA molecule capable of reducing the expression of a SusC gene endogenous to the fiber producing plant, and the RNA molecule comprising a first and second RNA region wherein
(ii) the first RNA region comprises a nucleotide sequence of at least 19 consecutive nucleotides comprising at least about 94% sequence identity to the nucleotide sequence of an endogenous SusC gene or to the nucleotide sequence according to claim 5 or the nucleotide sequence of any one of SEQ ID Nos.: 1, 4, 6, 8, 10, 12 or 14;
(iii) the second RNA region comprises a nucleotide sequence complementary to the at least 19 consecutive nucleotides of the first RNA region;
(iv) the first and second RNA region are capable of base-pairing to form a double stranded RNA molecule between at least the 19 consecutive nucleotides of the first and second region; and
(c) a 3′ end region comprising transcription termination and polyadenylation signals functioning in cells of the plant.

11. The expression cassette of claim 9, wherein the plant expressible promoter controls transcription in fiber cells of a fiber producing plant.

12. An expression cassette comprising the following operably linked DNA regions:
(a) a plant expressible promoter;
(b) a DNA region which upon introduction and transcription in a plant cell is processed into a miRNA, whereby the miRNA is capable of recognizing and guiding the cleavage of the mRNA of an endogenous SusC encoding gene of the plant; and
(c) optionally, a 3′ DNA region involved in transcription termination and polyadenylation.

13. The expression cassette of claim 12, wherein said DNA region comprises a nucleotide sequence which is essentially complementary to a nucleotide sequence of at least 21 consecutive nucleotides of a SusC encoding gene comprising the nucleotide sequence of any one of SEQ ID Nos.: 1, 4, 6, 8, 10, 12 or 14 provided that one or more of following mismatches are allowed:
(a) a mismatch between the nucleotide at the 5′ end of the miRNA and the corresponding nucleotide sequence in the RNA molecule;
(b) a mismatch between any one of the nucleotides in position 1 to position 9 of the miRNA and the corresponding nucleotide sequence in the RNA molecule; or
(c) three mismatches between any one of the nucleotides in position 12 to position 21 of the miRNA and the corresponding nucleotide sequence in the RNA molecule provided that there are no more than two consecutive mismatches.

14. A plant cell comprising a heterologous plant expressible promoter operably linked to a DNA molecule comprising:
(a) a DNA encoding a sucrose synthase according to claim 1;
(b) a DNA comprising the nucleotide sequence of any one of SEQ ID Nos.: 1, 4, 6, 8, 10, 12 or 14;
(c) a DNA which when transcribed results in an RNA molecule said RNA molecule comprising a nucleotide sequence of at least 19 consecutive nucleotides comprising at least about 94% sequence identity to the nucleotide sequence of an endogenous sucrose synthase isoform C encoding gene or to the complement of said nucleotides sequence of an endogenous sucrose synthase isoform C encoding gene;
(d) a DNA which when transcribed results in an RNA molecule said RNA molecule comprising a nucleotide sequence a nucleotide sequence of at least 19 consecutive nucleotides comprising at least about 94% sequence identity to the nucleotide sequence of any one of SEQ ID Nos.: 1, 4, 6, 8, 10, 12 or 14;
(e) a DNA, which when transcribed yields a double-stranded RNA molecule capable of reducing the expression of a SusC gene endogenous to the fiber producing plant, and the RNA molecule comprising a first and second RNA region wherein
(i) the first RNA region comprises a nucleotide sequence of at least 19 consecutive nucleotides comprising at least about 94% sequence identity to the nucleotide sequence of an endogenous SusC gene or the nucleotide sequence of any one of SEQ ID Nos.: 1, 4, 6, 8, 10, 12 or 14;
(ii) the second RNA region comprises a nucleotide sequence complementary to the at least 19 consecutive nucleotides of the first RNA region; and
(iii) the first and second RNA region are capable of base-pairing to form a double stranded RNA molecule between at least the 19 consecutive nucleotides of the first and second region; or
(f) a DNA which upon transcription in a plant cell is processed into a miRNA, whereby the miRNA is capable of recognizing and guiding the cleavage of the mRNA of an endogenous SusC encoding gene of the plant.

15. The plant cell of claim 14, wherein said DNA molecule operably linked to said plant expressible promoter is stably integrated in the genome of said plant cell.

16. The plant cell according to claim 14, wherein said cell is from a fiber producing plant.

17. The plant cell according to claim 16, wherein said cell is from cotton.

18. A plant comprising the plant cell of claim 14.

19. The plant according to claim 18, which is a fiber-producing plant.

20. The plant according to claim 18, which is a cotton plant.

21. A seed or progeny plant of the plant according to claim 18.

22. Fibers from the plant according to claim 20.

23. A method for modifying the fiber characteristics of a fiber-producing plant comprising modifying the functional level of a sucrose synthase isoform C or a sucrose synthase with similar characteristics in cells or cell walls or apoplastic fluid of said plant.

24. The method according to claim 23, wherein the functional level of a sucrose synthase isoform C or a sucrose synthase with similar characteristics is increased.

25. The method according to claim 24, wherein the functional level of a sucrose synthase isoform C is increased by providing the plants with an expression cassette comprising the following operably linked DNA molecules:
(A) a plant expressible promoter;
(B) a DNA encoding a sucrose synthase comprising
(i) an amino acid sequence comprising at least 80% sequence homology to the amino acid sequence of any one of SEQ ID Nos.: 2, 3, 5, 7, 9, 11 or 13;
(ii) an amino acid sequence comprising the amino acid sequence of any one of SEQ ID Nos.: 2, 3, 5, 7, 9, 11 or 13;
(iii) an amino acid sequence located in the amino-terminal part of said protein, said amino acid sequence comprising at least about 60% sequence identity to the amino acid sequence of SEQ ID No. 16; or
(iv) an amino acid sequence located at the carboxy-terminal part of said protein, said amino acid sequence comprising at least about 60% sequence identity to the amino acid sequence of SEQ ID No. 15;
(c) and, optionally, a transcription termination and polyadenylation region.

26. The method according to claim 24, wherein the functional level of a sucrose synthase similar to sucrose synthase isoform C is increased by providing the plants with an expression cassette comprising a plant expressible promoter operably linked to a DNA region encoding a sucrose synthase having a hydrophobic N-terminal region.

27. The method according to claim 23, wherein the functional level of a sucrose synthase isoform C or a sucrose synthase with similar characteristics is decreased.

28. The method according to claim 27, wherein the functional level of a sucrose synthase isoform C is decreased by providing the plants with an expression cassette comprising the following operably linked DNA molecules:
(A) a plant expressible promoter;
(B) a DNA which when transcribed results in an RNA molecule said RNA molecule comprising:
(i) a nucleotide sequence of at least 19 consecutive nucleotides comprising at least about 94% sequence identity to the nucleotide sequence of an endogenous sucrose synthase isoform C encoding gene or to the complement of said nucleotide sequence of an endogenous sucrose synthase isoform C encoding gene;
(ii) a nucleotide sequence of at least 19 consecutive nucleotides comprising at least about 94% sequence identity to a nucleotide sequence an isolated DNA molecule;
(iii) a nucleic acid encoding the sucrose synthase protein comprising
(a) an amino acid sequence comprising at least 80% sequence homology to the amino acid sequence of any one of SEQ ID Nos.: 2, 3, 5, 7, 9, 11 or 13;
(b) an amino acid sequence comprising the amino acid sequence of any one of SEQ ID Nos.: 2, 3, 5, 7, 9, 11 or 13;
(c) an amino acid sequence located in the amino-terminal part of said protein, said amino acid sequence comprising at least about 60% sequence identity to the amino acid sequence of SEQ ID No. 16;
(d) an amino acid sequence located at the carboxy-terminal part of said protein, said amino acid sequence comprising at least about 60% sequence identity to the amino acid sequence of SEQ ID No. 15; or
(iii) the nucleotide sequence of any one of SEQ ID Nos.: 1, 4, 6, 8, 10, 12 or 14; and
(C) optionally, a transcription termination and polyadenylation region.

29. The method of claim 23, wherein one of the following fiber characteristics has been modified: strength, length, fiber fineness, fiber maturity ratio, immature fiber content, fiber uniformity, or micronaire.

30. (canceled)

31. (canceled)

32. Fibers obtained by the method according to claim 23.

33. A tissue or a yarn made from the fibers according to claim 32.

34. A method for increasing UDP-glucose and fructose concentration in the apoplast of a plant cell, comprising providing said plant cell with an expression cassette according to claim 7.

35. The expression cassette of claim 10, wherein the plant expressible promoter controls transcription in fiber cells of a fiber producing plant.

36. The plant cell of claim 14, wherein said DNA molecule operably linked to said plant expressible promoter is stably integrated in the nuclear genome of said plant cell.

37. The method according to claim 27, wherein the functional level of a sucrose synthase isoform C is decreased by providing the plants with an expression cassette comprising the following operably linked DNA molecules:
(A) a plant expressible promoter;
(B) a DNA, which when transcribed yields a double-stranded RNA molecule capable of reducing the expression of a SusC gene endogenous to the fiber producing plant, and the RNA molecule comprising a first and second RNA region wherein
(i) the first RNA region comprises a nucleotide sequence of at least 19 consecutive nucleotides comprising at least about 94% sequence identity to the nucleotide sequence of an endogenous SusC gene or to the nucleotide sequence comprising
(a) an amino acid sequence comprising at least 80% sequence homology to the amino acid sequence of any one of SEQ ID Nos.: 2, 3, 5, 7, 9, 11 or 13;
(b) an amino acid sequence comprising the amino acid sequence of any one of SEQ ID Nos.: 2, 3, 5, 7, 9, 11 or 13;
(c) an amino acid sequence located in the amino-terminal part of said protein, said amino acid sequence comprising at least about 60% sequence identity to the amino acid sequence of SEQ ID No. 16;
(d) an amino acid sequence located at the carboxy-terminal part of said protein, said amino acid sequence comprising at least about 60% sequence identity to the amino acid sequence of SEQ ID No. 15; or
(e) the nucleotide sequence of any one of SEQ ID Nos.: 1, 4, 6, 8, 10, 12 or 14;
(ii) the second RNA region comprises a nucleotide sequence complementary to the at least 19 consecutive nucleotides of the first RNA region;
(iii) the first and second RNA region are capable of base-pairing to form a double stranded RNA molecule between at least the 19 consecutive nucleotides of the first and second region; and
(C) a 3′ end region comprising transcription termination and polyadenylation signals functioning in cells of the plant.

38. The expression cassette of claim 37, wherein the plant expressible promoter controls transcription in fiber cells of a fiber producing plant.

39. The method according to claim 27, wherein the functional level of a sucrose synthase isoform C is decreased by providing the plants with an expression cassette comprising the following operably linked DNA molecules:
(a) a plant expressible promoter;
(b) a DNA region which upon introduction and transcription in a plant cell is processed into a miRNA, whereby the miRNA is capable of recognizing and guiding the cleavage of the mRNA of an endogenous SusC encoding gene of the plant; and
(c) optionally, a 3′ DNA region involved in transcription termination and polyadenylation.

40. The method of claim 39, wherein said DNA region of said expression cassette comprises a nucleotide sequence which is essentially complementary to a nucleotide sequence of at least 21 consecutive nucleotides of a SusC encoding gene comprising the nucleotide sequence of any one of SEQ ID Nos.: 1, 4, 6, 8, 10, 12 or 14 provided that one or more of following mismatches are allowed:
(a) a mismatch between the nucleotide at the 5′ end of the miRNA and the corresponding nucleotide sequence in the RNA molecule;
(b) a mismatch between any one of the nucleotides in position 1 to position 9 of the miRNA and the corresponding nucleotide sequence in the RNA molecule; or
(c) three mismatches between any one of the nucleotides in position 12 to position 21 of the miRNA and the corresponding nucleotide sequence in the RNA molecule provided that there are no more than two consecutive mismatches.

41. A tissue or yarn made from the fibers according to claim 22.


SEQUENCE LISTING (55 sequences)

SEQ ID NO: 1 - DNA, 2635 bp, Gossypium hirsutum
  CDS (84)..(2474)
acgcggggtgtctttgatttttatccatatcctaattcttcatacattccttcccttctgccatttcaggaacccaaaaaacaatggcttcaatcagtgtttgtgagcgtttgggtgaatctctagctactcatccacagcaggcaaagtctatcttgtcaaggattgaaagcctcggaaagggtattcataagtctcaaaagctgctctcggttctcgataaagaggccggaaatcaagcacttgatgggatggtggtgggggtcctcaggtccactcaggaagctgtagtgtcgtctccattggttgcccttgccattcgttcagctcctggagtttgggagtacattgctgtggaggtccaaaagctttttgtggaggaaatgcccgttgctgagtatctacggttgaaggaagaacttgttgatggaagctccaatggcgagtttatgttggaattggactttggtgcattcaataattctgttcctcgtccatctctttcaaagtccattggtaatggcatggacttcctcaaccgccacctttctgccaagctatttcaagacaaggagaacttgaacttgttgcttgaatttctccaaattcactgccagaagggaaagggtatgctgttgaatgacagaatccaagatgtgaattccctccaacatgcattaaggaaggccgaggagtatctgactcctctatcctcggataccccgtactcagttttcgagaaaaggtttctggggattggtttggagaagggatggggtgataatgctgagcatgttcttgagatgatccatcttctattggatctccttcaggcacctgatcctgtcgcacttgaaagcttccttggaagaatcccattggtcgccaatgttgtaatcatgactccccatggatactttgcccaagacaatgttttgggataccctgacacaggtggtcaggttgtttatatcttagatcaagttcgtgccttggaggaagagttgcttcatcgtttcaagctgcaaggactcgacattaccccacgtatcctagtcattactcggctcctccctgatgctgtaggaacaacttgcggtcagcgtcttgagaaagtgtatggaaccaagtattctgatattcttcgggtacccttcagaactgagaagggaattgtacgtccatggatctcacgattcaaagtctggccttacctggagacttacactaaggatgttgcagctgagatcaccaaagagttccagggcaagcctgatctgattgttgggaactacagtgatggaaacattgttgcttctttattggcacataagtttgatgttacacagtgcactattgctcgtgcactcgagaagacgaaatacccggattcagacattaactggaaacagcttgaggataagtatcacttctcctgtcagtttactgctgatcttattgctatgaaccatactgattttatcatcaccagcaccttccaagagattgctggaagcaaggacactctcggccaatacgagagtcacattgctttcactcttccagggctctaccgcgttgttgatgggatcgatgtttttgatcccaaattcaatattgtctcccctggtgctgatatgagcatatacttcccttacacggaagagaagcggaggttgaagaagttccacccggagattgaagagcttctttacagccctgttgagaatacagagcacttatgtgtactaaaagaccgcaacaagccgattctgtttaccatggcaaggttggaccgagtgaagaacttaactgggctcgtagagttctatgccaagaacagccggctgagggaactggttaacttggttgtagtaggtggagataggagaaaggaatccaaggacttagaagaaaaggctgaaatgaagaagatgtatgaacttatcgaaaaatacaagttgaatggacaattcagatggatatcgtcccagatgaaccgagtgagaaatggtgaactctatcgttatatttgcgacaccaagggagcattcgttcagcctcctatatacgaggcttttggcttgactgttgttgaggcaatgacctgtggactaccaacatttgcaacatgttacgggggccctgctgagattatagttcacggaaaatcggggttcaacatcgatccttataacggtgatttggctgccgagacccttgccaatttcttcgagaagtgcaaagcggatccatcttattgggatgagatctcccagggagggttgaaacgcatacaggagaagtatacatggcagatttactccgagaagctattgactctcaccggagtttatggcttttcgaaacatgtagcttaccaggagcaacgtgggcgcaagcgttacattgaaatgttgcatgcatggatgtataacaatcgggtcaagactgttccactagctgttgagtaaagaaaaagcaaaaggctgcttggaaaccaaggaatttccaagaatgattgctattgaagttttatttttgcttctttttttcaatttcgtaaatgtttaatttcaactttggggtttccccttggaatgttttgaactctgggtttggtcccattgattgc

SEQ ID NO: 2 - protein, 796 aa, Gossypium hirsutum
MASISVCERLGESLATHPQQAKSILSRIESLGKGIHKSQKLLSVLDKEAGNQALDGMVVGVLRSTQEAVVSSPLVALAIRSAPGVWEYIAVEVQKLFVEEMPVAEYLRLKEELVDGSSNGEFMLELDFGAFNNSVPRPSLSKSIGNGMDFLNRHLSAKLFQDKENLNLLLEFLQIHCQKGKGMLLNDRIQDVNSLQHALRKAEEYLTPLSSDTPYSVFEKRFLGIGLEKGWGDNAEHVLEMIHLLLDLLQAPDPVALESFLGRIPLVANVVIMTPHGYFAQDNVLGYPDTGGQVVYILDQVRALEEELLHRFKLQGLDITPRILVITRLLPDAVGTTCGQRLEKVYGTKYSDILRVPFRTEKGIVRPWISRFKVWPYLETYTKDVAAEITKEFQGKPDLIVGNYSDGNIVASLLAHKFDVTQCTIARALEKTKYPDSDINWKQLEDKYHFSCQFTADLIAMNHTDFIITSTFQEIAGSKDTLGQYESHIAFTLPGLYRVVDGIDVFDPKFNIVSPGADMSIYFPYTEEKRRLKKFHPEIEELLYSPVENTEHLCVLKDRNKPILFTMARLDRVKNLTGLVEFYAKNSRLRELVNLVVVGGDRRKESKDLEEKAEMKKMYELIEKYKLNGQFRWISSQMNRVRNGELYRYICDTKGAFVQPPIYEAFGLTVVEAMTCGLPTFATCYGGPAEIIVHGKSGFNIDPYNGDLAAETLANFFEKCKADPSYWDEISQGGLKRIQEKYTWQIYSEKLLTLTGVYGFSKHVAYQEQRGRKRYIEMLHAWMYNNRVKTVPLAVE

SEQ ID NO: 3 - protein, 796 aa, Gossypium hirsutum
MASISVCERLGESLATHPQQAKSILSRIESLGKGIHKSQKLLSVLDKEAGNQALDGMVVEVLRSTQEAVVSSPLVALAIRSAPGVWEYIAVEVQKLFVEEMPVAEYLRLKEELVDGSSNGEFMLELDFGAFNNSVPRPSLSKSIGNGMDFLNRHLSAKLFQDKENLNLLLEFLQIHCQKGKGMLLNDRIQDVNSLQHALRKAEEYLTPLSSDTPYSVFEKRFLGIGLEKGWGDNAEHVLEMIHLLLDLLQAPDPVALESFLGRIPLVANVVIMTPHGYFAQDNVLGYPDTGGQVVYILDQVRALEEELLHRFKLQGLDITPRILVITRLLPDAVGTTCGQRLEKVYGTKYSDILRVPFRTEKGIVRPWISRFKVWPYLETYTKDVAAEITKEFQGKPDLIVGNYSDGNIVASLLAHKFDVTQCTIAHALEKTKYPDSDINWKQLEDKYHFSCQFTADLIAMNHTDFIITSTFQEIAGSKDTLGQYESHIAFTLPGLYRVVDGIDVFDPKFNIVSPGADMSIYFPYTEEKRRLKKFHPEIEELLYSPVENTEHLCVLKDRNKPILFTMARLDRVKNLTGLVEFYAKNSRLRELVNLVVVGGDRRKESKDLEEKAEMKKMYELIEKYKLNGQFRWISSQMNRVRNGELYRYICDTKGAFVQPPIYEAFGLTVVEAMTCGLPTFATCYGGPAEIIVHGKSGFNIDPYNGDLAAETLANFFEKCKADPSYWDEISQGGLKRIQEKYTWQIYSEKLLTLTGVYGFSKHVAYQEQRGRKRYIEMLHAWMYNNRVKTVPLAVE

SEQ ID NO: 4 - DNA, 3353 bp, Gossypium hirsutum
  misc_feature (32)..(34): startcodon
cccttctgccatttcaggaacccaaaaaacaatggcttcaatcagtgtttgtgagcgtttgggtgaatctctagctactcatccacagcaggcaaagtctatcttgtcaaggtataatgtaaataagtgctgccttcaaatttctggtctttcttcgttattttttattacattgtttatatatgtcatattgcaggattgaaagcctcggaaagggtattcataagtctcaaaagctgctctcggttctcgataaagaggccggaaatcaagcacttgatgggatggtggtggaggtcctcaggtccactcaggtcacaataaaacaaaccctttctttggttatgtttcatgatgagaatattggactgatatgttaccgttatctaataggaagctgtagtgtcgtctccattggttgcccttgccattcgttcagctcctggagtttgggagtacattgctgtggaggtccaaaagctttttgtggaggaaatgcccgttgctgagtatctacggttgaaggaagaacttgttgatggaaggtaggtagttttctcttacagctcctggtttatattatgtttgagtttatgttgaacaatgctcaatgatgtggttcagctccaatggcgagtttatgttggaattggactttggtgcattcaataattctgttcctcgtccatctctttcaaagtccattggtaatggcatggacttcctcaaccgccacctttctgccaagctatttcaagacaaggagaacttgaacttgttgcttgaatttctccaaattcactgccagaagggaaaggtaaaagtttcactttcactatttgtttttcctttctgtatacatcaaaacttaagaatcgttcaatgttatgccacagggtatgctgttgaatgacagaatccaagatgtgaattccctccaacatgcattaaggaaggccgaggagtatctgactcctctatcctcggataccccgtactcagttttcgagaaaaggtttctggggattggtttggagaagggatggggtgataatgctgagcatgttcttgagatgatccatcttctattggatctccttcaggcacctgatcctgtcgcacttgaaagcttccttggaagaatcccattggtcgccaatgttgtaatcatgactccccatggatactttgcccaagacaatgttttgggataccctgacacaggtggtcaggttgtttatatcttagatcaagttcgtgccttggaggaagagttgcttcatcgtttcaagctgcaaggactcgacattaccccacgtatcctagtcgtgagtatctgaatgttaattcagtttcgatcattatgtttaatgcttaatttggctgtttgtgtaccagattactcggctcctccctgatgctgtaggaacaacttgcggtcagcgtcttgagaaagtgtatggaaccaagtattctgatattcttcgggtacccttcagaactgagaagggaattgtacgtccatggatctcacgattcaaagtctggccttacctggagacttacactaaggatgttgcagctgagatcaccaaagagttccagggcaagcctgatctgattgttgggaactacagtgatggaaacattgttgcttctttattggcacataagtttgatgttacacaggtttgttaaataccaattaattgaacaagttccttgctgtattttgatgttccccgtgcaatctaaaaacttggttttcaatgttttatgcagtgcactattgctcatgcactcgagaagacgaaatacccggattcagacattaactggaaacagcttgaggataagtatcacttctcctgtcagtttactgctgatcttattgctatgaaccatactgattttatcatcaccagcaccttccaagagattgctggaaggtaatctatctcccactttactacatttggttcccttgtacgttgtaaatgttgccacctatttgagtccctaacaatgttttcattccatgattttcagcaaggacactctcggccaatacgagagtcacattgctttcactcttccagggctctaccgcgttgttgatgggatcgatgtttttgatcccaaattcaatattgtctcccctggtgctgatatgagcatatacttcccttacacggaagagaagcggaggttgaagaagttccacccggagattgaagagcttctttacagccctgttgagaatacagagcacttgtgagattttggtcctatttttacacccttttcactagattttgtgttcagtgttaacaatgtcttaaattgttgtctcacagatgtgtactaaaagaccgcaacaagccgattctgtttaccatggcaaggttggaccgagtgaagaacttaactgggctcgtagagttctatgccaagaacagccggctgagggaactggttaacttggttgtagtaggtggagataggagaaaggaatccaaggacttagaagaaaaggctgaaatgaagaagatgtatgaacttatcgaaaaatacaagttgaatggacaattcagatggatatcgtcccagatgaaccgagtgagaaatggtgaactctatcgttatatttgcgacaccaagggagcattcgttcagcctcctatatacgaggcttttggcttgactgttgttgaggcaatgacctgtggactaccaacatttgcaacatgttacgggggccctgctgagattatagttcacggaaaatcggggttcaacatcgatccttataacggtgatttggctgccgagacccttgccaatttcttcgagaagtgcaaagcggatccatcttattgggatgagatctcccagggagggttgaaacgcatacaggagaagtatacatggcagatttactccgagaagctattgactctcaccggagtttatggcttttcgaaacatgtagcttaccaggagcaacgtgggcgcaagcgttacattgaaatgttgcatgcatggatgtataacaatcgggtaaaacaatcatgtctattattattattacatcaaacatcattttactctttcattgaactgaaattttcatgtttctgaatgtttgtttaattgtgtgctttacaggtcaagactgttccactagctgttgagtaaagaaaaagcaaaaggctgcttggaaaccaaggaatttccaagaatgattgctattgaagttttatttttgcttctttttttcaatttcgtaaatgtttaatttcaactttggggtttccccttggaatgttttgaactctgggtttggtcccatt

SEQ ID NO: 5 - protein, 796 aa, Gossypium barbadense
MASISVCERLGESLATHPQQAKSILSRIESLGKGIHKSQKLLSVLDKEAGNQALDGMVVEVLRSTQEAVVSSPLVALAIRSAPGVWEYIAVEVQKLFVEEMPVAEYLRLKEELVDGSSNGEFMLELDFGAFNNSVPRPSLSKSIGNGMDFLNRHLSAKLFQDKESLNLLLEFLQIHCQKGKGMLLNDRIQDVNSLQHALRKAEEYLTPLSSDTPYSVFEKRFLGIGLEKGWGDNAEHVLEMIHLLLDLLQAPDPVALESFLGRIPLVANVVIMTPHGYFAQYNVLGYPDTGGQVVYILDQVRALEEELLHRFKLQGLDITPRILVITRLLPDAVGTTCGQRLEKVYGTKYSDILRVPFRTEKGIVRPWISRFKVWPYLETYTKDVAAEITKEFQGKPDLIVGNYSDGNIVASLLAHKFDVTQCTIAHALEKTKYPDSDINWKQLEDKYHFSCQFTADLIAMNHTDFIITSTFQEIAGSKDTLGQYESHIAFTLPGLYRVVDGIDVFDPKFNIVSPGADMSIYFPYTEEKRRLKKFHPEIEELLYSPVENTEHLCVLKDRNKPILFTMARLDRVKNLTGLVEFYAKNSRLRELVNLVVVGGDRRKESKDLEEKAEMKKMYELIEKYKLNGQFRWISSQMNRVRNGELYRYICDTKGAFVQPAIYEAFGLTVVEAMTCGLPTFATCYGGPAEIIVHGKSGFNIDPYNGDLAAETLANFFEKCKADPSYWDEISQGGLKRIQEKYTWQIYSEKLLTLTGVYGFSKHVAYQEQRGRKRYIEMLHAWMYNNRVKTVPLAVE

SEQ ID NO: 6 - DNA, 3352 bp, Gossypium barbadense
  misc_feature (32)..(34): start codon
cccttctgccatttcaggaacccaaaaaacaatggcttcaatcagtgtttgtgagcgtttgggtgaatctttagctactcatccacagcaggcaaagtctatcttgtcaaggtataatgtaaataagtgctgccttcaaatttctggtctttcttcgttattttttattacattgtttatatatgtcatattgcaggattgaaagcctcggaaagggtattcataagtctcaaaagctgctctcggttctcgataaagaggccggaaatcaagcacttgatgggatggtggtggaggtcctcaggtccactcaggtcacaataaaacaaaccctttctttggttatgtttcatgatgagaatattggactgatatgttaccgttatctaataggaagctgtagtgtcgtctccattggttgcccttgccattcgttcagctcctggagtttgggagtacattgctgtggaggtccaaaagctttttgtggaggaaatgcccgttgctgagtatctacggttgaaggaagaacttgttgatggaaggtaggtagttttctcttacagctcctggtttatgttatgtttgagtttatgttgaacaatgctcaatgatgtggttcagctccaatggcgagtttatgttggaattggactttggtgcattcaataattctgttcctcgtccatctctttcaaagtccattggtaatggcatggacttcctcaaccgccacctttctgccaagctatttcaagacaaggagagcttgaacttgttgcttgaatttctccaaattcactgccagaagggaaaggtaaaagtttcactttcactatttgtttttcctttctgtatacatcaaaacttaagaatcgttcaatgttatgccacagggtatgctgttgaatgacagaatccaagatgtgaattccctccaacatgcattaaggaaggccgaggagtatctgactcctctatcctcggataccccgtactcagttttcgagaaaaggtttctggggattggtttggagaagggatggggtgataatgctgagcatgttcttgagatgatccatcttctattggatctccttcaggcacctgatcctgtcgcacttgaaagcttccttggaagaatcccattggtcgccaatgttgtaatcatgactccccatggatactttgcccaatacaatgttttgggataccctgacacaggtggtcaggttgtttatatcttagatcaagttcgtgccttggaggaagagttgcttcatcgtttcaagctgcaaggactcgacattaccccacgtatcctagtcgtgagtatctgaatgttaattcagtttcgatcattatgtttaatgcttaatttggctgtttgtgtaccagattactcggctcctccctgatgctgtaggaacaacttgcggtcagcgtcttgagaaagtgtatggaaccaagtattctgatattcttcgggtacccttcagaactgagaagggaattgtacgtccatggatctcacgattcaaagtctggccttacctggagacttacactaaggatgttgcagctgagatcaccaaagagttccagggcaagcctgatctgattgttgggaactacagtgatggaaacattgttgcttctttattggcacataagtttgatgttacacaggtttgttaaataccaattaattgaacaagttccttgctgtattttgatgttccccgtgcaatctaaaaacttggttttcaatgttttatgcagtgcactattgctcatgcactcgagaagacgaaatacccggattcagacattaactggaaacagcttgaggataagtatcacttctcctgtcagtttactgctgatcttattgctatgaaccatactgattttatcatcaccagcaccttccaagagattgctggaaggtaatctatctcccactttactacatttggttcccttgtacgttgtaaatgttgccacctatttgagtccctaacaatgttttcattccatgattttcagcaaggacactctcggccaatacgagagtcacattgctttcactcttccagggctctaccgcgttgttgatgggatcgatgtttttgatcccaaattcaatattgtctcccctggtgctgatatgagcatatacttcccttacacggaagagaagcggaggttgaagaagttccacccggagattgaagagcttctttacagccctgttgagaatacagagcacttgtgagattttggtcctatttttacaccctttcactagattttgtgttcagtgttaacaatgtcttaaattgttgtctcacagatgtgtactaaaagaccgcaacaagccgattctgtttaccatggcaaggttggaccgagtgaagaacttaactgggctcgtagagttctatgccaagaacagccggctgagggaactggttaacttggttgtagtaggtggagataggagaaaggaatccaaggacttagaagaaaaggctgaaatgaagaagatgtatgaacttatcgaaaaatacaagttgaatggacaattcagatggatatcgtcccagatgaaccgagtgagaaatggtgaactctatcgttatatttgcgacaccaagggagcattcgttcagcctgctatatacgaggcttttggcttgactgttgttgaggcaatgacctgtggactaccaacatttgcaacatgttacgggggccctgctgagattatagttcacggaaaatcggggttcaacatcgatccttataacggtgatttggctgccgagacccttgccaatttcttcgagaagtgcaaagcggatccatcttattgggatgagatctcccagggagggttgaaacgcatacaggagaagtatacatggcagatttactccgagaagctattgactctcaccggagtttatggcttttcgaaacatgtagcttaccaggagcaacgtgggcgcaagcgttacattgaaatgttgcatgcatggatgtataacaatcgggtaaaacaatcatgtctattattattattacatcaaacatcattttactctttcattgaactgaaaatttcatgtttctgaatgtttgtttaattgtgtgctttacaggtcaagactgttccactagctgttgagtaaagaaaaagcaaaaggctgcttggaaaccaaggaatttccaagaatgattgctattgaagttttatttttgcttctttttttcaatttggtaaatgtttaatttcaactttggggtttccccttggaatgttttgaactctgggtttggtcccatt

SEQ ID NO: 7 - protein, 796 aa, Gossypium arboreum
MASISVCERLGESLATHPQQAKSILSRIESLGKGIHKSQKLLSVLDKEAGNQALDGMVVEVLRSTQEAVVSSPLVALAIRSAPGVWEYIAVEVQKLFVEEMPVAEYLRLKEELVDGSSNGEFMLELDFGAFNNSVPRPSLSKSIGNGMDFLNRHLSAKLFQDKENLSLLLEFLQIHCQKGKGMLLNDRIQDVNSLQHALRKAEEYLTPLTSDTPYSVFEKRFREIGLEKGWGDNAEHVLEMIHLLLDLLQAPDPVALESFLGTIPLVANVVIMTPHGYFAQDNVLGYPDTGGQVVYILDQVRALEEELLHRFKLQGLDITPRILVITRLLPDAVGTTCGQRLEKVYGTKYSDILRVPFRTEKGIVRPWISRFKVWPYLETYTKDVAAEVTKEFQGKPDLIVGNYSDGNIVASLLALKFDVTQCTIAHALEKTKYPDSDINWKQLEDKYHFSCQFTADLIAMNHTDFIITSTFQEIAGSKDTLGQYESHIAFTLPGLYRVVDGIDVFDPKFNIVSPGADMSIYFPYTEEKRRLKKFHPEIEELLYSPVENTEHLCVLKDRNKPVLFTMARLDRVKNLTGLVEFYAKNSRLRELVNLVVVGGDRRKESKDLEEKAEMKKMYELIEKYKLNGQLRWISSQMNRVRNGELYRYICDTKGAFVQPAIYEAFGLTVVEAMTCGLPTFATCYGGPAEIIVHGKSGFNIDPYNGDLAAETLANFFEKCKADPSYWDEISQGGLKRIQEKYTWQIYSEKLLTLTGVYGFSKHVAYQEQRGRKRYIEMLHALMYNNRVKTVPLAVE

SEQ ID NO: 8 - DNA, 3352 bp, Gossypium arboreum
  exon (32)..(111): exon1
cccttctgccatttcaggaaccaaaaaagcaatggcttcaatcagtgtctgtgagcgtttgggtgaatctctagctactcatccacagcaggcaaagtctatcttgtcaaggtataatgtaaataagtgctgccttcaaagttctggtctttctttgttattttttgttacattgtttatatatgtcatattgcaggattgaaagccttggaaagggtattcataagtctcaaaagctgctctcagttctcgataaagaggccggaaatcaagcacttgatgggatggtggtagaggtcctcaggtccactcaggtcacaataaaacaaaccctttctttggttatgtatgatgagaatattggactgatatgttaccattatctataggaagctgtagtgtcgtctccattggttgcccttgccattcgttcagctcctggagtttgggagtacattgctgtggaggtccaaaagctttttgtggaggaaatgcccgttgctgagtatctacggttgaaggaagaacttgttgatggaaggtaggtagttttctcttacagctcctggtttatgttatgtttgagtttatgttgaacaatgctcaatgatgtggttcagctccaatggcgagtttatgttggaattggactttggtgcattcaataattctgttcctcgtccatctctttcaaagtccattggtaatggcatggacttcctcaaccgccacctttctgccaagctatttcaagacaaggagaacttgagcttgttgcttgaatttctccaaattcactgccagaagggaaaggtaaaagtttcactttcactatttgtttttcctttctgtatacatcaaaacttaagaatcgttcaatgttatgccacagggtatgctgttgaatgacagaatccaagatgtgaattccctccaacatgcattgaggaaggccgaggagtatctgactcctctaacgtcggataccccgtactcggttttcgagaaaaggtttcgggagattggtttggagaagggatggggtgataatgctgagcatgttcttgagatgatccatcttctattggatctccttcaggcacctgatcctgtcgcacttgaaagcttccttggaacaatcccattggtcgccaatgttgtaatcatgactccccatggatactttgcccaagacaatgttttgggataccctgacacaggtggtcaggttgtttatatcttagatcaagttcgtgccttggaggaagagttgcttcatcgtttcaagctgcaaggactcgacattaccccacgaatcctagtcgtgagtatctgaatattaattcggtttcgatcattatgtttaatgcttaatttggctgtttatgtaccagattactcggctcctccctgatgctgtaggaacaacttgcggtcagcgtcttgagaaagtgtatggaaccaagtattctgatattcttcgggtacccttcagaactgagaagggaattgtacgtccatggatctcacgattcaaagtctggccttacctggagacttacactaaggatgttgcagctgaggtcaccaaagagttccagggcaaacctgatctgattgttgggaactacagtgatggaaacattgttgcttctttattggcactgaagtttgatgttacacaggtttgttaaatgccaattaattgaacaagttcctttctgtattttgatttttcccgtgcaatctaaaaacttggttttcaatgttttatgcagtgcactattgctcatgcacttgagaagacgaaatacccggattcagacattaactggaaacaacttgaggataagtatcacttctcctgtcagtttactgctgatcttattgctatgaaccatactgattttatcatcaccagcaccttccaagagattgctggaaggtaatctatctcccactttactacatttggttcccttgtacgttgtaaatgttgccacctatttgagtccctaacaatgtttccattccatgattttcagcaaggacactctcggccaatacgagagtcacatcgctttcactcttccagggctctaccgcgttgttgatgggatcgatgtttttgatcccaaattcaatattgtctcccctggtgctgatatgagcatatacttcccttacacggaagagaagcgaaggttgaagaagttccacccggagattgaagagcttctttacagccctgttgagaatacagagcacttgtaagattttggtcctagttttacacccgttttcaccggattttgtgttcagtgttaacaatgtcttaaattgttgtctcacagatgtgtactaaaagaccgcaacaagccggttctgtttaccatggcaaggttggaccgagtgaagaacttaactgggctcgtagagttctatgccaagaacagccggctgagggaactggttaacttggttgtagtaggtggagataggagaaaggaatccaaggacttagaagaaaaggctgaaatgaagaagatgtatgaacttatcgaaaaatacaagttgaatggacaattgagatggatatcgtcccagatgaaccgtgtgagaaatggtgaactctatcgttatatttgcgacaccaagggagcattcgttcagcctgctatatacgaggcttttggcttgactgttgttgaggcaatgacctgtggactaccaacatttgcaacatgttacgggggccctgctgagattatagttcacggaaaatcggggttcaacatcgacccttataacggtgatttggctgccgagacccttgccaatttcttcgagaaatgcaaagcggatccatcttattgggatgagatctcccagggagggttgaaacgcatacaggagaagtatacatggcagatttactccgagaagctattgactctcaccggagtttatggcttttcgaaacatgtagcttaccaggagcaacgtgggcgcaagcgttacattgaaatgttgcatgcactgatgtataacaatcgggtaaaacagtcatgtctattattattattacatcaaacatcattttactctttcattgaactgaaaatttcatatttctgaatgtttaattgtgtgctttacaggtcaagactgttcctctagctgttgagtaaagaaaaagcaaaaagctgcttggaaactaaggaatttccaagaatgactgctgttgaagttttatttttgcttttattattttttttttcaattggtaaatgtttaatttcaactttgggtttccccttggaatgtttcgaactctgggtttggtcccatt

SEQ ID NO: 9 - protein, 796 aa, Gossypium raimondii
MASISVCERLGESLATHPQQAKSILSRIESLGKGIHKSQKLLSVLDKEAGNQALDGMVVEVLRSTQEAVVSSPLVALAIRSAPGVWEYIALEFQKLFVEEMPVAEYLRLKEELVDGSSNGEFMLELDFGAFNNSVPRPSLSKSIGNGMDFLNRHLSAKLFQDKENLNLLLEFLQIRCQKGKGMLLNDRIQDVNSLQHALRKAEEYLTPLSSDTPYSVFEKRFLGIGLEKGWGDNAEHVLEMIHLLLDLLQAPDPVALESFLGRIPLVANVVIMTPHGYFAQDNVLGYPDTGGQVVYILDQVRALEEELLHRFKLQGLDITPRILVITRLLPDAVGTTCGQRLEKVYGTKYSDILRVPFRTEKGIVRPWISRFKVWPYLETYTKDVAAEITKEFQGKPDLIVGNYSDGNIVASLLAHKFDVTQCSIAHALEKTKYPDSDINWKQLEDKYHFSCQFTADLIAMNHTDFIITSTFQEIAGSKDTLGQYESHIAFTLPGLYRVVDGIDVFDPKFNIVSPGADMSIYFPYTEEKRRLKKFHPEIEELLYSPVENTEHLCVLKDRNKPILFTMARLDRVKNLTGLVEFYAKNSRLRELVNLVVVGGDRRKESKDLEEKAEMKKMYELIEKYKLNGQFRWISSQMNRVRNGELYRYICDTKGAFVQPAIYEAFGLTVVEAMTCGLPTFATCYGGPAEIIVHGKSGFNIDPYNGDLAAETLANFFEKCKADPSYWDEISQGGLKRIQEKYTWQIYSEKLLTLTGVYGFSKHVAYQEQRGRKRYIEMLHALMYNNRVKTAPLAVE

SEQ ID NO: 10 - DNA, 3354 bp, Gossypium raimondii
  exon (32)..(111): exon1
cccttctgccatttcaggaacctaaaaaacaatggcttcaatcagtgtttgtgagcgtttgggtgaatctctagctactcatccacagcaggcaaagtctatcttgtcaaggtataatgtaaataagtgctgccttcaaatttctggtctttctttgttattttttattacattgtttatatatgtcatattgcaggattgaaagcctcggaaagggtattcataagtctcaaaagctgctctcggttctcgataaagaggccggaaatcaagcacttgatgggatggtggtggaggtcctcaggtccactcaggtcacaataaaacaaaccctttctttggttatgtttcatgatgagaatattggactgatatgttaccgctatctaataggaagctgtagtgtcgtctccattggttgcccttgccattcgttcagctcctggagtttgggagtacattgctctggagttccaaaagctttttgtggaggaaatgcccgttgctgagtatctacggttgaaggaagaacttgttgatggaaggtaggtagttttctcttacagctcctggtttatattatgtttgagtttatgttgaacaatgctcaatgatgtggttcagctccaatggcgagtttatgttggaattggactttggtgcattcaataattctgttcctcgtccatctctttcaaagtccattggtaatggcatggacttcctcaaccgccacctttctgccaagctatttcaagacaaggagaacttgaacttgttgcttgaatttctccaaattcgctgccagaagggaaaggtaaaagtttcactttcactatttgtttttcctttctgtatacatcaaaacttaagaatcgttcaatgttatgccacagggtatgctgttgaatgacagaatccaagatgtgaattccctccaacatgcattgaggaaggccgaggagtatctgactcctctatcgtcggataccccgtactcagttttcgagaaaaggtttctggggattggtttggagaagggatggggcgataatgctgagcatgttcttgagatgatccatcttctattggatctccttcaggcacctgatcctgtcgcacttgaaagcttccttggaagaatcccattggtcgccaatgttgtaatcatgactccccatggatactttgcccaagacaatgttttgggataccctgacacaggtggtcaggttgtttatatcttagatcaagttcgtgccttggaggaagagttgcttcatcgtttcaagctgcaaggactcgacattaccccacgtatcctagtcgtgagtatctgaatgttaattcagtttcgatcattatgtttaatgcttaatttggctgtttgtgtaccagattactcggctcctccctgatgctgtaggaacaacttgcggacagcgtcttgagaaagtgtatggaaccaagtattctgatattcttcgggtacccttcagaactgagaagggaattgtacgtccatggatctcacgattcaaagtctggccttacctggagacttacactaaggatgttgcagctgagatcaccaaagagttccagggcaagcctgatctgattgttgggaactacagtgatggaaacattgttgcttctttattggcacataagtttgatgttacacaggtttgttaaatgccaattaattgaacaagttccttgctgtattttgatgttcctcgtgcaatctaaaaacttggttttcaatgttttatgcagtgctctattgctcatgcactcgagaagacgaaatacccggattcagacattaactggaaacagcttgaggataagtatcacttctcctgtcagtttactgctgatcttattgctatgaaccatactgattttatcatcaccagcaccttccaagagattgctggaaggtaatctatctcccactttactacatttggttcccttgtacgttgtaaatgttgccacctatttgagtccctaacaatgttttcattccatgattttcagcaaggacactctcggccaatacgagagtcacattgctttcactcttccaggtctctaccgcgttgttgatgggatcgatgtttttgatcccaaattcaatattgtctcccctggtgctgatatgagcatatacttcccttacacggaagagaagcggaggttgaagaagttccacccggagattgaagagcttctttacagccctgttgagaatacagagcacttgtgagattttggtcctatttttacacccttttcactagattttgtgttcagtgttaacaatgtcttaaattgttgtctcacagatgtgtactaaaagaccgcaacaagccgattctgtttaccatggcaaggttggaccgagtgaagaacttaactgggctcgtagagttctatgccaagaacagccggctgagggaactggttaacttggttgtagtaggtggagataggagaaaggaatccaaggacttagaagaaaaggctgaaatgaagaagatgtatgaacttatcgaaaaatacaagttgaatggacaattcagatggatatcgtcccagatgaaccgtgtgagaaatggtgaactctatcgttatatttgcgacaccaagggagcattcgttcagcctgctatatacgaggcttttggcttgactgttgttgaggcaatgacctgtggactaccaacatttgcaacatgctacgggggccctgctgagattatagttcacggaaaatcggggttcaacatcgatccttataacggtgatttggctgccgagacccttgccaatttcttcgagaagtgcaaagcggatccatcttattgggatgagatctcccagggagggttgaaacgcatacaggagaagtatacatggcagatttactccgagaagctattgactctcaccggagtttatggcttttcgaaacatgtagcttaccaggagcaacgtgggcgcaagcgttacattgaaatgttgcatgcattgatgtataacaatcgggtaaaacaatcatgtctattattattattacatcaaacatcattttactctttcattgaactgaaaatttcatgtttctgaatgtttgtttaattgtgtgttttacaggtcaagactgctccactagctgttgagtaaagaaaaagcaaaaggctgcttggaaactaaggaatttccaagaatgattactattgaagttttatttttgcttcttttttttcaatttggtaaatgtttaatttcaactttggggtttccccttggaatgttttgaactctgggtttggtcccatt

SEQ ID NO: 11 - protein, 796 aa, Gossypium longicalyx
MASISVCERLGESLATHPQQAKSILSRIESLGKGIHKSQKLLSVLDKEAGNQALDGMVVEVLRSTQEAVVSSPLVALAIRSAPGVWEYIAVEVQKLFVEEMPVAEYLRLKEELVDGSSKGEFMLELDFGAFNNSVPRPSLSKSIGNGMDFLNRHLSAKLFQDKENLNLLLEFLQIHCQKGKGMLLNDRIQDVNSLQHALRKAEEYLTPLTSDTPYSVFEKRFREIGLEKGWGDNAEHVLEMIHLLLDLLQAPDPVALESFLGRIPLVANVVIMTPHGYFAQDNVLGYPDTGGQVVYILDQVRALEEELLHRFKLQGLDITPRILVITRLLPDAVGTTCGQRLEKVYGTKYSDILRVPFRTEKGIVRPWISRFKVWPYLETYTKDVAAEITKEFQGKPDLIVGNYSDGNIVASLLAHKFDVTQCTIAHALEKTKYPDSDINWKQLEDKYHFSCQFTADLIAMNHTDFVITSTFQEIAGSKDTLGQYESHIAFTLPGLYRVVDGIDVFDPKFNIVSPGADMSIYFPYTEEKRRLKKFHPEIEELLYSPVENTEHLCVLKDRNKPVLFTMARLDRVKNLTGLVEFYAKNSRLRELVNLVVVGGDRRKESKDLEEKAEMKKMYELIEKYKLNGQFRWISSQMNRVRNGELYRYICDTKGAFVQPAIYEAFGLTVVEAMTCGLPTFATCYGGPAEIIVHGKSGFNIDPYNGDLAAETLANFFEKCKADPSYWDEISQGGLKRIQEKYTWQIYSEKLLTLTGVYGFSKHVAYQEQRGRKRYIEMLHALMYNNRVKTVPLAVE

SEQ ID NO: 12 - DNA, 3353 bp, Gossypium longicalyx
  exon (32)..(111): exon 1
cccttctgccatttcaggaacctaaaaaacaatggcttcaatcagtgtctgtgagcgtttgggtgaatctctagctactcatccacagcaggcaaagtctatcttgtcaaggtataatgtaaataagtgctgccttcaaagttctggtctttctttgttattttttattacattgtttatatgtcatattgcaggattgaaagccttggaaagggtattcataagtctcaaaagctgctctcggttctcgataaagaggccggaaatcaagcacttgatgggatggtggtggaggtcctcaggtccactcaggtcacaataaaacaaaccctttctttggttatgtttcatgatgagaatattggactgatatgttaccattatctaataggaagctgtagtgtcgtctccattggttgcccttgccattcgttcagctcctggagtttgggagtacattgctgtggaggtccaaaagctttttgtggaggaaatgcccgttgctgagtatctacggttgaaggaagaacttgttgacggaaggtaggtagttttctcttacagctcctggtttatgttatgtttgagtttatgttgaacaatgctcaatgatgtggttcagctccaagggcgagtttatgttggaattggactttggtgcattcaataattctgttcctcgtccatctctttcaaagtccattggtaatggcatggacttcctcaaccgccacctttctgccaagctatttcaagacaaggagaacttgaacttgttgcttgaatttctccaaattcactgccagaagggaaaggtaaaagtttcactttcactatttgtttttcctttatacatcaaaactcaagaatcgttcaatgttatgccacagggtatgctgttgaatgacagaatccaagatgtgaattccctccaacatgcattgaggaaggccgaggagtatctgactcccctaacgtcggataccccgtactcggttttcgagaaaaggtttcgggagattggtttggagaagggatggggtgataatgctgagcatgttcttgagatgatccatcttctattggatctccttcaggcacctgatcctgtcgcacttgaaagcttccttggaagaatcccattggtcgccaatgttgtaatcatgactccccatggatactttgctcaagacaatgttttgggataccctgacacaggtggtcaggttgtttatatcttagatcaagttcgtgccttggaggaagagttgcttcatcgtttcaagctgcaaggactcgacattaccccacgtatcctagtcgtgagtatctgaatgttaattcggtttcgatcattatgtttaatgcttaatttggctgtttatgtaccagattactcggctcctccctgatgctgtaggaacaacttgcggtcagcgtcttgaaaaagtgtatggaaccaagtattctgatattcttcgggtacccttcagaactgagaagggaattgtacgtccatggatctcacgattcaaagtctggccttacctggagacttacactaaggatgttgcagctgagatcaccaaagagttccagggcaagcctgatctgattgttgggaactacagtgatggaaacattgttgcttctttattggcacataagtttgatgttacacaggtttgttaaatgccaattaattgaacaagttccttgctgtattttgatgtttcccgtgcaatctaaaaacttggttttcaatgttttatgcagtgcactattgctcatgcacttgagaagacgaaatacccggattcagacattaactggaaacagcttgaggataagtatcacttctcctgtcagtttactgctgatcttattgctatgaaccatactgattttgtcatcaccagcaccttccaagagattgctggaaggtaatctatctcccactttactaaatttggttcccttgtacgttgtaaatgttgccacctatttgagtccctaacaatgtttccattccatgattttcagcaaggacactctcggccaatacgagagtcacattgctttcactcttccagggctctaccgcgttgttgatgggatcgatgtttttgatcccaaattcaatattgtctcccctggtgctgatatgagcatatacttcccttacacggaagagaagcgaaggttgaagaagttccacccggagattgaagagcttctttacagccctgttgagaatacagagcacttgtgggattttggtcctatttttacaccctttttcactagattttgtgttcagtgttaacaatgtcttgaattgttgtctcacagatgtgtactaaaagaccgcaacaagccggttctgtttaccatggcaaggttggaccgagtgaagaacttaactgggctcgtagagttctatgccaagaacagccggctgagggaactggttaacttggttgtagtaggtggagataggagaaaggaatccaaggacttagaagaaaaggctgaaatgaagaagatgtatgaacttatcgaaaaatacaagttgaatggacaattcagatggatatcgtcccagatgaaccgtgtgagaaatggtgaactctatcgttatatttgcgacaccaagggagcattcgttcagcctgctatatacgaggcttttggcttgactgttgttgaggcaatgacctgtggactaccaacatttgcaacatgttacgggggccctgctgagattatagttcacggaaaatcggggttcaacatcgacccttataacggtgatttggctgccgagaccctcgccaatttcttcgagaaatgcaaagcggatccatcttattgggatgagatctcccagggagggttgaaacgcatacaggagaagtatacatggcagatttactccgagaagctattgactctcaccggagtttatggcttttcgaaacatgtagcttaccaggagcaacgtgggcgcaagcgttacattgaaatgttgcatgcattgatgtataacaatcgggtaaaacaatcatgtctattattattattacatcaaacatcattttactctttcattgaactgaaaatttcatgtttctgaatgtttaattgtgtgctttacaggtcaagactgttcctctagctgttgagtaaagaaacagcaaaaagctgcttggaaaccaaggaatttccaagaatgattgctattgaagttttatttttgcttttctttttttttttttttttcaatttggtaaatgtttaatttcaactttggggtttccccttggaatgttttgaactctgggtttggtccc

SEQ ID NO: 13 - protein, 796 aa, Gossypium longicalyx
MASISVCERLGESLATHPQQAKSILSRIESLGKGIHKSQKLLSVLDKEAGNQALDGMVVEVLRSTQEAVVSSPLVALAIRSAPGVWEYIAVEVQKLFVEEMPVAEYLRLKEELVDGSSNGEFMLELDFGAFNNSVPRPSLSKSIGNGMDFLNRHLSAKLFQDKENLNLLLEFLQIHCQKGKGMLLNDRIQDVNSLQHALRKAEEYLTPLSSDTPYSVFEKRFLGIGLEKGWGDNAEHVLEMIHLLLDLLQAPDPVALESFLGRIPLVANVVIMTPHGYFAQDNVLGYPDTGGQVVYILDQVRALEEELLHRSKLQGLDITPRILVITRLLPDAVGTTCGQRLEKVYGTKYSDILRVPFRTEKGIVRPWISRFKVWPYLETYTKDVAAEITKEFQGKPDLIVGNYSDGNIVASLLAHKFDVTQCTIAHALEKTKYPDSDINWKQLEDKYHFSCQFTADLIAMNHTDFIITSTFQEIAGSKDTLGQYESHIAFTLPGLCRVVDGIDVFDPKFNIVSPGADMSIYFPYTEEKRRLKKFHPEIEELLYSPVENTEHLCVLKDRNKPILFTMARLDRVKNLTGLVEFYAKNSRLRELVNLVVVGGDRRKESKDLEEKAEMKKMYELIEKYKLNGQFRWISSQMNRVRNGELYRYICDTKGAFVQPPIYEAFGLTVVEAMTCGLPTFATCYGGPAEIIVHGKSGFNIDPCNGDLAAETLANFFEKCKADPSYWDEISQGGLKRIQEKYTWQIYSEKLLTLTGVYGFSKHVAYQEQRGRKRYIEMLHAWMYNNRVKTVPLAVE

SEQ ID NO: 14 - DNA, 3353 bp, Gossypium longicalyx
  exon (32)..(111): exon 1
cccttctgccatttcaggaacccaaaaaacaatggcttcaatcagtgtttgtgagcgtttgggtgaatctctagctactcacccacagcaggcaaagtctatcttgtcaaggtataatgtaaataagtgctgccttcaaatttctggtctttcttcgttattttttattacattgtttatatatgtcatattgcaggattgaaagcctcggaaagggtattcataagtctcaaaagctgctctcggttctcgataaagaggccggaaatcaagcacttgatgggatggtggtggaggtcctcaggtccactcaggtcacaataaaacaaaccctttctttggttatgtttcatgatgagaatattggactgatatgttaccgttatctaataggaagctgtagtgtcgtctccattggttgcccttgccattcgttcagctcctggagtttgggagtacattgctgtggaggtccaaaagctttttgtggaggaaatgcccgttgctgagtatctacggttgaaggaagaacttgttgatggaaggtaggtagttttctcttacagctcctggtttatattatgtttgagtttatgttgaacaatgctcaatgatgtggttcagctccaatggcgagtttatgttggaattggactttggtgcattcaataattctgttccccgtccatctctttcaaagtccattggtaatggcatggacttcctcaaccgccacctttctgccaagctatttcaagacaaggagaacttgaacttgttgcttgaatttctccaaattcactgccagaagggaaaggtaaaagtttcactttcactatttgtttttcctttctgtatacatcaaaacttaagaatcgttcaatgttatgccacagggtatgctgttgaatgacagaatccaagatgtgaattccctccaacatgcattaaggaaggccgaggagtatctgactcctctatcctcggataccccgtactcagttttcgagaaaaggtttctggggattggtttggagaagggatggggtgataatgctgagcatgttcttgagatgatccatcttctattggatctccttcaggcacctgatcctgtcgcacttgaaagcttccttggaagaatcccattggtcgccaatgttgtaatcatgactccccatggatactttgcccaagacaatgttttgggataccctgacacaggtggtcaggttgtttatatcttagatcaagttcgtgccttggaggaagagttgcttcatcgttccaagctgcaaggactcgacattaccccacgtatcctagtcgtgagtatctgaatgttaattcagtttcgatcattatgtttaatgcttaatttggctgtttgtgtaccagattactcggctcctccctgatgctgtaggaacaacttgcggtcagcgtcttgagaaagtgtatggaaccaagtattctgatattcttcgggtacccttcagaactgagaagggaattgtacgtccatggatctcacgattcaaagtctggccttacctggagacttacactaaggatgttgcagctgagatcaccaaagagttccagggcaagcctgatctgattgttgggaactacagtgatggaaacattgttgcttctttattggcacataagtttgatgttacacaggtttgttaaataccaattaattgaacaagttccttgctgtattttgatgttccccgtgcaatctaaaaacttggttttcaatgttttatgcagtgcactattgctcatgcactcgagaagacgaaatacccggattcagacattaactggaaacagcttgaggataagtatcacttctcctgtcagtttactgctgatcttattgctatgaaccatactgattttatcatcaccagcaccttccaagagattgctggaaggtaatctatctcccactttactacatttggttcccttgtacgttgtaaatgttgccacctatttgagtccctaacaatgttttcattccatgattttcagcaaggacactctcggccaatacgagagtcacattgctttcactcttccagggctctgccgcgttgttgatgggatcgatgtttttgatcccaaattcaatattgtctcccctggtgctgatatgagcatatacttcccttacacggaagagaagcggaggttgaagaagttccacccggagattgaagagcttctttacagccctgttgagaatacagagcacttgtgagattttggttctatttttacacccttttcactagattttgtgttcagtgttaacaatgtcttaaattgttgtctcacagatgtgtactaaaagaccgcaacaagccgattctgtttaccatggcaaggttggaccgagtgaagaacttaactgggctcgtagagttctatgccaagaacagccggctgagggaactggttaacttggttgtagtaggtggagataggagaaaggaatccaaggacttagaagaaaaggctgaaatgaagaagatgtatgaacttatcgaaaaatacaagttgaatggacaattcagatggatatcgtcccagatgaaccgagtgagaaatggtgaactctatcgttatatttgcgacaccaagggagcattcgttcagcctcctatatacgaggcttttggcttgactgttgttgaggcaatgacctgtggactaccaacatttgcaacatgttacgggggccctgctgagattatagttcacggaaaatcggggttcaacatcgatccttgtaacggtgatttggctgccgagacccttgccaatttcttcgagaagtgcaaagcggatccatcttattgggatgagatctcccagggagggttgaaacgcatacaggagaagtatacatggcagatttactccgagaagctattgactctcaccggagtttatggcttttcgaaacatgtagcttaccaggagcaacgtgggcgcaagcgttacattgaaatgttgcatgcatggatgtataacaatcgggtaaaacaatcatgtctattattattattacatcaaacatcattttactctttcattgaactgaaattttcatgtttctgaatgtttgtttaattgtgtgctttacaggtcaagactgttccactagctgttgagtaaagaaaaagcaaaaggctgcttggaaaccaaggaatttccaagaatgattgctattgaagttttatttttgcttctttttttcaatttcgtaaatgtttaatttcaactttggggtttccccttggaatgttttgaactctgggtttggtcccatt

SEQ ID NO: 15 - protein, 26 aa, Artificial Sequence
  C-terminal consensus sequence SusC
AYQEQRGRKRYIEMLHAWMYNNRVKT

SEQ ID NO: 16 - protein, 24 aa, Artificial Sequence
  N-terminal consensus sequence SusC
HKSQKLLSVLDKEAGNQALDGMVV

SEQ ID NO: 17 - DNA, 22 bp, Artificial Sequence
  SusC specific PCR primer 1
cccttctgccatttcaggaacc

SEQ ID NO: 18 - DNA, 22 bp, Artificial Sequence
  SusC specific PCR primer 2
aatgggaccaaacccagagttc

SEQ ID NO: 19 - protein, 21 aa, Artificial Sequence
  Chemically Synthesized
RKAEEYLTPLSSDTPYSVFEK

SEQ ID NO: 20 - protein, 14 aa, Artificial Sequence
  Chemically Synthesized
KLLTLTGVYGFSKH

SEQ ID NO: 21 - protein, 10 aa, Artificial Sequence
  Chemically Synthesized
KYTWQIYSEK

SEQ ID NO: 22 - protein, 13 aa, Artificial Sequence
  Chemically Synthesized
RIQDVNSLQHALR

SEQ ID NO: 23 - protein, 21 aa, Artificial Sequence
  Chemically Synthesized
RKAEEYLTPLSSDTPYSVFEK

SEQ ID NO: 24 - protein, 13 aa, Artificial Sequence
  Chemically Synthesized
KNLTGLVEFYAKN

SEQ ID NO: 25 - protein, 18 aa, Artificial Sequence
  Chemically Synthesized
RSTQEAVVSSPLVALAIR

SEQ ID NO: 26 - protein, 11 aa, Artificial Sequence
  Chemically Synthesized
KLQGLDITPRI

SEQ ID NO: 27 - protein, 13 aa, Artificial Sequence
  Chemically Synthesized
RVVDGIDVFDPKF

SEQ ID NO: 28 - protein, 14 aa, Artificial Sequence
  Chemically Synthesized
KLLTLTGVYGFSKH

SEQ ID NO: 29 - protein, 21 aa, Artificial Sequence
  Chemically Synthesized
KDTLGQYESHIAFTLPGLYRV

SEQ ID NO: 30 - protein, 15 aa, Artificial Sequence
  Chemically Synthesized
KLFVEEMPVAEYLRL

SEQ ID NO: 31 - protein, 14 aa, Artificial Sequence
  Chemically Synthesized
KLLTLTGVYGFSKH

SEQ ID NO: 32 - protein, 13 aa, Artificial Sequence
  Chemically Synthesized
RIQDVNSLQHALR

SEQ ID NO: 33 - protein, 15 aa, Artificial Sequence
  Chemically Synthesized
RYIEMLHAWMYNNRV

SEQ ID NO: 34 - protein, 15 aa, Artificial Sequence
  Chemically Synthesized
RLGESLATHPQQAKS

SEQ ID NO: 35 - protein, 15 aa, Artificial Sequence
  Chemically Synthesized
KLFVEEMPVAEYLRL

SEQ ID NO: 36 - protein, 21 aa, Artificial Sequence
  Chemically Synthesized
KDTLGQYESHIAFTLPGLYRV

SEQ ID NO: 37 - protein, 21 aa, Artificial Sequence
  Chemically Synthesized
RKAEEYLTPLSSDTPYSVFEK

SEQ ID NO: 38 - protein, 25 aa, Artificial Sequence
  Chemically Synthesized
KSGFNIDPYNGDLAAETLANFFEKC

SEQ ID NO: 39 - protein, 13 aa, Artificial Sequence
  Chemically Synthesized
KNLTGLVEFYAKN

SEQ ID NO: 40 - protein, 18 aa, Artificial Sequence
  Chemically Synthesized
RSTQEAVVSSPLVALAIR

SEQ ID NO: 41 - protein, 21 aa, Artificial Sequence
  Chemically Synthesized
KDTLGQYESHIAFTLPGLYRV

SEQ ID NO: 42 - protein, 15 aa, Artificial Sequence
  Chemically Synthesized
RYIEMLHAWMYNNRV

SEQ ID NO: 43 - protein, 13 aa, Artificial Sequence
  Chemically Synthesized
KTKYPDSDINWKQ

SEQ ID NO: 44 - protein, 13 aa, Artificial Sequence
  Chemically Synthesized
KSIGNGMDFLNRH

SEQ ID NO: 45 - protein, 15 aa, Artificial Sequence
  Chemically Synthesized
RLGESLATHPQQAKS

SEQ ID NO: 46 - protein, 11 aa, Artificial Sequence
  Chemically Synthesized
RALEEELLHRF

SEQ ID NO: 47 - protein, 4 aa, Artificial Sequence
  Chemically Synthesized
RXXS

SEQ ID NO: 48 - protein, 60 aa, Artificial Sequence
  Uridine-binding region on the sucrose synthase C proteins
VVIMTPHGYFAQDNVLGYPDTGGQVVYILDQVRALEEELLHRFKLQGLDITPRILVITRL

SEQ ID NO: 49 - protein, 12 aa, Artificial Sequence
  putative sysyC actin-binding site
KDVAAEITKEFQ

SEQ ID NO: 50 - protein, 12 aa, Artificial Sequence
  consensus sequence for the site in between different plant sucrose synthases
XDXXXEXXXEXX

SEQ ID NO: 51 - protein, 24 aa, Artificial Sequence
  N-terminal region of sysyC gene
HKSQKLLSVLDKEAGNQALDGMVV

SEQ ID NO: 52 - protein, 26 aa, Artificial Sequence
  C-terminal region of sysyC gene
AYQEQRGRKRYIEMLHAWMYNNRVKT

SEQ ID NO: 53 - protein, 178 aa, Artificial Sequence
  GhsusyC SGC FM966 protein
MASISVCERLGESLATHPQQAKSILSRIESLGHGIMKSQKLLSVLDKEAGNQALDGNQALDGMVVEVLRSTQEAVVSSPLVALAIRSAPGVWEYIAVEVQKLFVEEMPVAEYLRLKEELVDGKYTWQTYSEKLLTLTGVYGFSKHVAYQEQRGRKRYIEMLHAWMYNNRVKTVPLAVE

SEQ ID NO: 54 - protein, 186 aa, Artificial Sequence
  GhsusyA SGD FM966 protein
MANPVITRVHSLRERLDETLLAHRNEILALLSRIEGKGKGIIQHHQIILEFEAIPEENRKKLADKKLADGAFFEVLKASQEAIVLPPWVALAVRPRPGVWEYIRVNVHALVVEELTVAEYLHFKEELVDGKYTWKIYSERLLTLTGVYGFWKHVSNLERRESRRYLEMFYALKYRKLAESVPLAEE

SEQ ID NO: 55 - protein, 187 aa, Artificial Sequence
  GhsusyB SGD FM966 protein
MAERVITRVHSLRERLDDTLIAHRNEVLALLTRIEGKGKGIIQHHQIILEFEAIPEETRKKLADRKKLADGAFSEILRSSQDEAIVLPPWVALAVRPRPGVWEYIKVNHALVVEELTVAEYLHFKEELVDGKYTWQIYSERLLTLTGVYGFWKHVSNLDRRESRRYLEMFYALKYRKLAESVPLAEE